(19) United States
(12) Patent Application Publication
(10) Pub. No.: US 2010/0056623 A1
(43) Pub. Date: Mar. 4, 2010

(54) FUMAGILLOL DERIVATIVES OR METHOD FOR PREPARATION OF FUMAGILLOL DERIVATIVES, AND PHARMACEUTICAL COMPOSITIONS COMPRISING THE SAME

(75) Inventors: Sang Joon Lee, Kyunggi-do (KR); Soon Kil Ahn, Seoul (KR); Hong Woo Lee, Kyeonggi-Do (KR); Joong Bok Ahn, Chungcheongnam-Do (KR); Jae Soo Shin, Seoul (KR); Young Min Kwon, Gyeonggi-Do (KR)

Correspondence Address: HESLIN ROTHENBERG FARLEY & MESITI PC, 5 COLUMBIA CIRCLE, ALBANY, NY, 12203, US

(73) Assignee: CHONG KUN DANG PHARMACEUTICAL CORP., Seoul (KR)

(21) Appl. No.: 11/814,809

(22) PCT Filed: Jan. 26, 2005
(86) PCT No.: PCT/KR05/00211

§ 371 Date: Oct. 25, 2007

(51) Int. Cl. A61K 31/336; C07D 305/14; A61P 35/00

(52) U.S. Cl. 514/475; 549/332

(57) ABSTRACT

The present invention relates to a fumagillol derivative, pharmaceutically acceptable salts thereof and a method for preparing the same. The compounds of the present invention can be prepared through acylation, hydrolysis and alkylation. The compound of the present invention can be prepared in the form of a pharmaceutically acceptable salt or inclusion compound. The present invention provides fumagillol derivatives having the following characteristics: increased inhibiting effect on angiogenesis, low toxicity, excellent solubility and chemical stability as compared to conventional angiogenesis inhibitors. The compounds of the present invention can be used as an anti-cancer medicine, inhibitor of cancer metastasis, or the therapeutic agent for treating rheumatic arthritis, psoriasis, diabetic retinitis or obesity.

### TECHNICAL FIELD

The present invention relates to a fumagillol derivative or a method for preparation thereof, and pharmaceutical compositions comprising the same.

### BACKGROUND ART

Angiogenesis is a phenomenon of generating new capillary vessels, which is one of the pathological phenomena happened in various diseases as well as one of normal physiological actions.

Additionally, Angiogenesis has a deep connection with growth and metastasis of solid cancer, rheumatic arthritis, diabetic retinopathy, psoriasis, or the like [Billington, D.C. Drug Design and Discovery, (1991), 8, 3.], and the compounds inhibiting angiogenesis show the effect of treatment for obesity [J. Folkman, PNAS, (2002), 99, 10730˜10735].

The compounds inhibiting angiogenesis have been developed and reported through many researches. Recently, as the clinical importance of therapeutic agents by means of controlling angiogenesis has been emphasized, researches on angiogenesis have increased. According to clinical results of anticancer medicines using angiogenesis inhibitors, in particular, it is expected that they cause little problems caused by general anticancer medicines, including adverse effect and tolerance. In other word, there are few possibilities that the problem of tolerance will occur since an angiogenesis inhibitor does not directly act on tumor cells, but acts on endotherial cells of a living organism. Additionally a synergistic anticancer effect is expected by a therapy in combination with conventional anticancer medicines that have been employed up to the present.

Presently, as the known patent inventions related to a fumagillol derivative, there are known O-chloroacetylcarbamoylfumagillol in EP-B1-357061, O-(4-dimethylaminoethoxycinnamoyl)fumagillol and O-(3,4,5-trimethoxycinnamoyl)fumagillol in U.S. Pat. No. 6,063,812A (which was filed an application by the present inventor). Although there is the strong point in which the angiogenesis inhibiting action of the above mentioned fumagillol derivative is excellent, there were problems that had to be improved with toxicity, chemical stability and solubility.

The compound of the present invention is for offering a fumagillol derivative or a method for preparation thereof, which inhibit angiogenesis and show lower toxicity, improvement of chemical stability and excellent solubility, in order to solve the conventional problem.

### DISCLOSURE OF THE INVENTION

The compounds of the present invention relate to a fumagillol derivative represented by the Chemical Formula 1 below, and a pharmaceutically acceptable salt thereof; and the a method for preparation thereof.

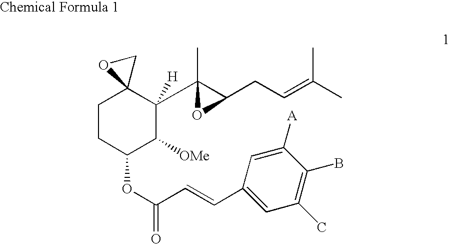

(Wherein,

A, B and C represent independently or simultaneously hydrogen, C₁-C₆ alkoxy, halogen, C₁-C₆ alkyl, trifluoromethyl, cyano, nitro, 4-hydroxymethylphenoxy, —XCH₂ₙOH or —XCH₂CH₂OₘCH₂CH₂OH, wherein X represents nitrogen or oxygen; n is 3, 4, 5 or 6; and m is 0, 1 or 2,

with proviso that at least one of above A, B, C is one substituent selected from the group consisting of 4-hydroxymethylphenoxy, —XCH₂ₙOH and —XCH₂CH₂OₘCH₂CH₂OH.)

The compounds of the present invention are, preferably,
O-(4-(2-hydroxyethoxy)cinnamoyl)fumagillol,
O-(3,5-dimethoxy-4-(2-hydroxyethoxy)cinnamoyl)fumagillol,
O-(4-(2-hydroxyethoxy)-3-methoxycinnamoyl)fumagillol,
O-(3-(2-hydroxyethoxy)-4-methoxycinnamoyl)fumagillol,
O-(4-(2-hydroxyethylamino)cinnamoyl)fumagillol,
O-(3-(2-hydroxyethylamino)cinnamoyl)fumagillol,
O-(4-chloro-3-(2-hydroxyethylamino)cinnamoyl)fumagillol,
O-(4-(4-hydroxymethylphenoxy)cinnamoyl)fumagillol,
O-(3,5-dimethoxy-4-(4-hydroxymethylphenoxy)cinnamoyl)fumagillol,
O-(4-(4-hydroxymethylphenoxy)-3-methoxycinnamoyl)fumagillol,
O-(3-(4-hydroxymethylphenoxy)-4-methoxycinnamoyl)fumagillol,
O-(4-(2-hydroxyethoxyethoxy)cinnamoyl)fumagillol,
O-(3,5-dimethoxy-4-(2-hydroxyethoxyethoxy)cinnamoyl)fumagillol,
O-(4-(2-hydroxyethoxyethoxy)-3-methoxycinnamoyl)fumagillol,
O-(3-(2-hydroxyethoxyethoxy)-4-methoxycinnamoyl)fumagillol,
O-(4-(2-hydroxyethoxyethylamino)cinnamoyl)fumagillol,
O-(3-(2-hydroxyethoxyethylamino)cinnamoyl)fumagillol,
O-(4-chloro-3-(2-hydroxyethoxyethylamino)cinnamoyl)fumagillol,
O-(4-(3-hydroxypropoxy)cinnamoyl)fumagillol,
O-(3-cyano-4-(3-hydroxypropoxy)cinnamoyl)fumagillol,
O-(4-(4-hydroxybutoxy)cinnamoyl)fumagillol,
O-(3-methyl-4-(4-hydroxybutoxy)cinnamoyl)fumagillol,
O-(4-(5-hydroxypentoxy)cinnamoyl)fumagillol,
O-(3-nitro-4-(5-hydroxypentoxy)cinnamoyl)fumagillol,
O-(4-(6-hydroxyhexyloxy)cinnamoyl)fumagillol,
O-(3-trifluoromethyl-4-(6-hydroxyhexyloxy)cinnamoyl)fumagillol or
O-(4-(2-hydroxyethoxyethoxyethoxy)cinnamoyl)fumagillol.

The compounds of the present invention are, more preferably,
O-(4-(2-hydroxyethoxy)cinnamoyl)fumagillol,
O-(3,5-dimethoxy-4-(2-hydroxyethoxy)cinnamoyl)fumagillol,
O-(4-(2-hydroxyethoxy)-3-methoxycinnamoyl)fumagillol,
O-(3-(2-hydroxyethoxy)-4-methoxycinnamoyl)fumagillol,
O-(4-(2-hydroxyethylamino)cinnamoyl)fumagillol,
O-(3-(2-hydroxyethylamino)cinnamoyl)fumagillol,
O-(4-chloro-3-(2-hydroxyethylamino)cinnamoyl)fumagillol or
O-(4-(3-hydroxypropoxy)cinnamoyl)fumagillol.

The compounds of the present invention are, most preferably,
O-(4-(2-hydroxyethoxy)cinnamoyl)fumagillol,
O-(3,5-dimethoxy-4-(2-hydroxyethoxy)cinnamoyl)fumagillol or
O-(4-(2-hydroxyethoxy)-3-methoxycinnamoyl)fumagillol.

The fumagillol derivatives of the present invention, which are represented by the Chemical Formula 1, may be prepared in the form of a pharmaceutically acceptable salt, and it can be prepared by using inorganic or organic acid.

Inorganic acids such as hydrochloric acid, hydrobromic acid, sulfuric acid, phosphoric acid or nitric acid may be used, and organic acids such as citric acid, acetic acid, lactic acid, tartaric acid, maleic acid, gluconic acid, succinic acid, formic acid, trifluoroacetic acid, oxalic acid, fumaric acid, methanesulfonic acid, benzenesulfonic acid, p-toluenesulfonic acid or camphorsulfonic acid may be used.

The fumagillol derivatives of the present invention, which are represented by the Chemical Formula 1, or salts thereof may be prepared in the form of inclusion compounds by using pharmaceutically acceptable cyclodextrin, and cyclodextrin such as hydroxypropyl-β-cyclodextrin or sulfobutylether-7-β-cyclodextrin may be used.

According to the preferred embodiment of the compounds of the present invention, the compounds represented by the Chemical Formula 1 can be prepared via acylation, hydrolysis and alkylation. The processes are explained by means of the Reaction Schemes here in below.

### (1) Acylation Step

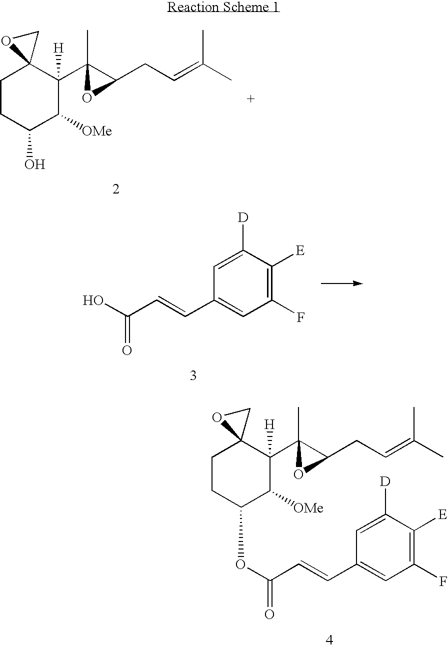

(Wherein,

D, E and F represent independently or simultaneously hydrogen, C₁-C₆ alkoxy, halogen, C₁-C₆ alkyl, trifluoromethyl, cyano, nitro, acetoxy, acetamino or 4-acetoxymethylphenoxy, with the proviso that at least one of above D, E and F is one substituent selected from the group consisting of acetoxy, acetamino and 4-acetoxymethylphenoxy.)

The acylation of the Reaction Scheme 1 may be performed by reacting a compound of the Chemical Formula 2, which is a starting material, with a substituted cinnamoyl acid derivatives of the Chemical Formula 3, or a reactive derivative thereof such as an acid anhydride, a mixed anhydride, an acid chloride, an acid p-toluenesulfonic anhydride, an acid mesylic anhydride, a 2-pyridine thiol ester or a phenyl ester, in the presence of a base.

The amount of the substituted cinnamoyl acid derivatives, which are represented by the Chemical Formula 3, or a reactive derivative thereof may be 1 to 5 equivalents, preferably 2 to 3 equivalents, relative to the amount of a compound of the Chemical Formula 2.

As a base used in the acylation, a tertiary amine such as triethyl amine, diisopropylethyl amine, pyridine and dimethylaminopyridine, or an alkaline metal hydride such as sodium hydride and potassium hydride may be used in an amount of 1 to 10 equivalents. Preferably, triethyl amine, or sodium hydride may be used in an amount of 4 to 6 equivalents as a base of the acylation.

As a solvent for the acylation, dimethylformamide, dimethylacetamide, dichloromethane, chloroform, tetrahydrofuran, diethylether, dioxane, acetonitrile, benzene or toluene etc. may be used, and dimethylformamide, toluene or dichloromethane is preferably used.

The reaction temperature of acylation is 0 to 50° C., preferably 20 to 30° C.

### (2) Hydrolysis Step

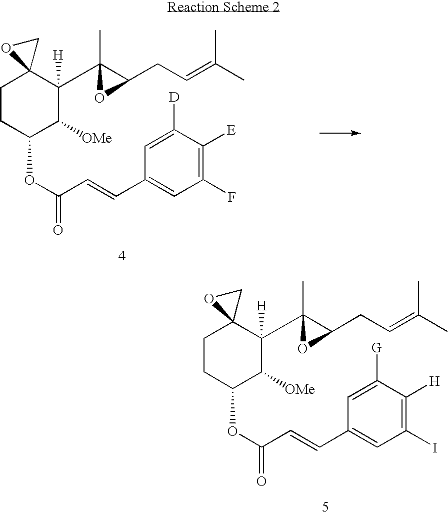

(Wherein,

D, E and F are the same as defined in the above;

G, H and I represent independently or simultaneously hydrogen, C₁-C₆ alkoxy, halogen, C₁-C₆ alkyl, trifluoromethyl, cyano, nitro, 4-hydroxymethylphenoxy, hydroxy or amine, with the proviso that at least one of above G, H and I is one substituent selected from the group consisting of 4-hydroxymethylphenoxy, hydroxy and amine.)

The hydrolysis may be performed by using a compound of the Chemical Formula 4, which is obtained by performing the acylation in the Reaction Scheme 1, and a common base. As a preferred base, potassium carbonate or cesium carbonate may be used in an amount of 1 to 5 equivalents, preferably 1 to 2 equivalents. As a solvent for the hydrolysis, methanol, ethanol, propanol, isopropanol, butanol or purified water may be used, and preferably, methanol or ethanol may be used. Then, the reaction temperature of hydrolysis is 0 to 50° C., preferably 20 to 30° C.

### (3) Alkylation Step

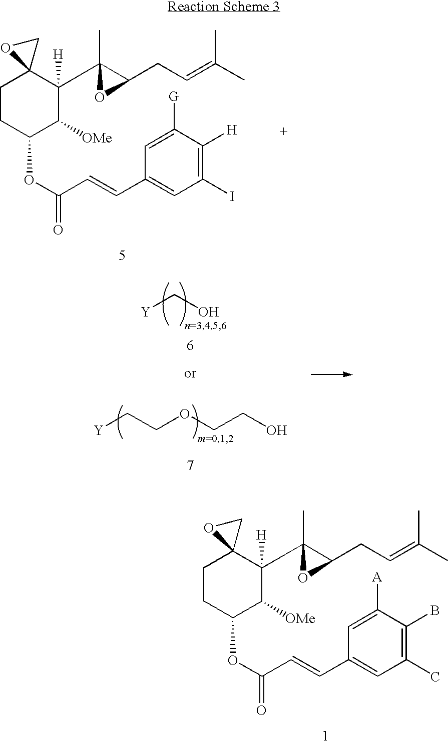

(Wherein,

A, B, C, G, H and I are the same as defined in the above;

Y represents halogen; n is 3, 4, 5 or 6; and m is 0, 1 or 2)

The alkylation may be performed by reacting a compound of the Chemical Formula 5, which is obtained by performing the hydrolysis in the Reaction Schemes 2, with a compound of the Chemical Formula 6 or a compound of the Chemical Formula 7. A compound of the Chemical Formula 6 is, for example, 3-chloropropanol, 4-chlorobutanol, 5-chloropentanol or 6-chlorohexanol, and a compound of the Chemical Formula 7 is, for example, 2-iodoethanol, 2-chloroethanol, 2-(2-chloroethoxy)ethanol or 2-(2-(2-chloroethoxy)ethoxy)ethanol, and they may be used in an amount of 1 to 10 equivalents, preferably 3 to 5 equivalents, relative to the amount of a compound of the Chemical Formula 5.

As a base used in the alkylation, potassium carbonate, sodium carbonate, cesium carbonate, calcium carbonate, sodium hydride or potassium hydride may be used, and potassium carbonate or sodium carbonate is preferably used in an amount of 5 to 7 equivalents.

As a solvent for the alkylation, dimethylformamide, dimethylacetamide, tetrahydrofuran or acetone may be used, and dimethylformamide is preferably used. Then, the reaction temperature of the alkylation is 50 to 100° C., preferably 80 to 100° C.

The present invention provides anti-cancer compositions that comprise a compound of the Chemical Formula 1 or the pharmaceutically acceptable salts thereof as an active ingredient, and a pharmaceutically acceptable carrier.

Since a compound of the Chemical Formula 1 or salts thereof has excellent angiogenesis-inhibiting effect, they can be used as an anticancer drugs or an inhibitor for a cancer metastasis, or as a therapeutic agent for treating rheumatic arthritis, psoriasis, diabetic retinitis and obesity.

The pharmaceutical compositions of the present invention may be prepared in the form of the preparation for oral administration, such as tablets, troches, lozenges, water soluble or oily suspensions, preparation powders or granulas, emulsions, hard or soft capsules and syrubs or elixirs.

In order to prepare in the form of tablets and capsules, binder such as lactose, sucrose, sorbitol, mannitol, starch, amylopectin, cellulose and gelatin, excipient such as dicalcium phosphate, disintegrant such as corn starch and sweet potato starch, and lubricant such as magnesium stearate, calcium stearate, sodium steargyl fumarate and polyethylene glycol wax may be contained.

In the case of capsule, liquid carriers such as fatty oils may be contained along with the above-mentioned materials.

In this case, sterilized aqueous solutions, non-aqueous solvents, suspensions, emulsions and lyophilizing agents are included in the preparation for administration. Vegetable oil such as propylene glycol, polyethylene glycol and olive oils, and injectable ester such as ethyl oleate may be used as non-aqueous solvents and solvents for suspension.

The active doses of a compound of the Chemical Formula 1 of the present invention are 0.2˜200 mg/kg, and it may be administrated in single or divided doses per day. However, the doses need to be varied, and it is specially varied according to the weight and the peculiarity of physical condition of patients, the type and seriousness of diseases, the property of preparation, and the property, period and frequency of the administration of medicine.

### BEST MODE

The compound of the present invention is described in more detail by referring to the examples below, but it should be noticed that the present invention is not restricted to the examples by any means.

### Example 1

### A preparation of O-(4-(2-hydroxyethoxy)cinnamoyl)fumagillol

Step 1: A preparation of O-(4-acetoxycinnamoyl)fumagillol

4-acetoxycinnamic acid (1.825 g, 8.85 mmol) in toluene (20 ml) was stirred, thionylchloride (1.29 ml, 1.77 mmol) was added dropwise thereto, and the resultant mixture was reflux-stirred for 4 hour. Then the solvent was removed by evaporation under reduced pressure, and the residue was dissolved in dimethylformamide (20 ml). Sodium hydride (850 mg, 21.25 mmol) and a compound of the Chemical Formula 2 (1.0 g, 3.54 mmol) were added dropwise thereto, and then the resultant mixture was stirred for 4 hour at ordinary temperature. The solution was added to an aqueous solution of saturated ammonium acetate (200 ml), and extracted with ethyl acetate (250 ml). The organic layer was washed 3 times with a saturated saline solution (200 ml). The organic layer was dried over anhydrous magnesium sulfate, filtered and then concentrated under reduced pressure. The residue was purified by column chromatography (ethyl acetate n-hexane=14) to obtain 775 mg (46%) of the title compound as colorless syrub.

¹H-NMR (400 MHz, CDCl₃) δ: 7.64 (d, 1H, J=16 Hz), 7.53 (m, 2H), 7.12 (m, 2H), 6.44 (d, 1H, J=16 Hz), 5.74 (m, 1H), 5.21 (m, 1H), 3.70 (dd, 1H, J=11.1, 2.7 Hz), 3.45 (s, 3H), 3.01 (d, 1H, J=4.4 Hz), 2.62 (t, 1H, J=6.4 Hz), 2.57 (d, 1H, J=4.4 Hz), 2.35 (m, 1H), 2.30 (s, 3H), 2.19-1.80 (m, 5H), 1.74 (s, 3H), 1.65 (s, 3H), 1.25 (s, 3H), 1.10 (m, 1H)

### Step 2: A preparation of O-(4-hydroxycinnamoyl)fumagillol

O-(4-acetoxycinnamoyl)fumagillol (770 mg, 1.636 mmol), which was obtained in the step 1, was dissolved in methanol (3 ml), and then potassium carbonate (226 mg, 1.636 mmol) was added thereto. The resultant mixture was stirred for 1 hour at ordinary temperature, and the reaction solution was added to an aqueous solution of saturated ammonium acetate (200 ml), and extracted with ethyl acetate (250 ml). The organic layer was washed 2 times with a saturated saline solution (200 ml). The organic layer was dried over anhydrous magnesium sulfate, filtered and concentrated under reduced pressure. The residue was purified by column chromatography (ethyl acetate:n-hexane=1:2) to obtain 384 mg (55%) of the title compound (55%) as a white solid.

¹H-NMR (400 MHz, CDCl₃) δ: 7.44 (d, 1H, J=16 Hz), 7.21 (d, 2H, J=8.6 Hz), 6.82 (d, 2H, J=8.6 Hz), 6.50 (brs, 1H), 6.00 (d, 1H, J=16 Hz), 5.76 (m, 1H), 5.21 (m, 1H), 3.74 (dd, 1H, J=11, 2.7 Hz), 3.50 (s, 3H), 3.01 (d, 1H, J=4.4 Hz), 2.70 (t, 1H, J=6.4 Hz), 2.58 (d, 1H, J=4.4 Hz), 2.41 (m, 1H), 2.20-1.87 (m, 5H), 1.87 (s, 3H), 1.75 (s, 3H), 1.27 (s, 3H), 1.06 (m, 1H)

### Step 3: A preparation of O-(4-(2-hydroxyethoxy)cinnamoyl)fumagillol

O-(4-hydroxycinnamoyl)fumagillol (150 mg, 0.35 mmol), which was obtained in the step 2, was dissolved in dimethylformamide (10 ml), and then potassium carbonate (290 mg, 2.10 mmol) and 2-iodoethanol (0.11 ml, 1.40 mmol) were added thereto. The resultant mixture was stirred for 6 hour at about 80° C. and cooled to ordinary temperature. The reaction solution was added to a saturated ammonium acetate (200 ml), and extracted with ethyl acetate (250 ml). The organic layer was washed 2 times with a saturated saline solution (200 ml). The organic layer was dried over anhydrous magnesium sulfate, filtered and concentrated under reduced pressure. The residue was purified by column chromatography (ethyl acetate:n-hexane=2:3) to obtain 95 mg (56%) of the title compound as a white solid.

¹H-NMR (400 MHz, CDCl₃) δ: 7.61 (d, 1H, J=16 Hz), 7.46 (m, 2H), 6.89 (m, 2H), 6.35 (d, 1H, J=16 Hz), 5.73 (m, 1H), 5.21 (m, 1H), 4.10 (m, 2H), 3.96 (m, 2H), 3.69 (dd, 1H, J=11, 2.7 Hz), 3.45 (s, 3H), 2.99 (d, 1H, J=4.4 Hz), 2.60 (t, 1H, J=6.4 Hz), 2.56 (d, 1H, J=4.4 Hz), 2.35 (m, 1H), 2.19-2.0 (m, 4H), 1.90 (m, 1H), 1.74 (s, 3H), 1.65 (s, 3H), 1.22 (s, 3H), 1.08 (m, 1H).

### Example 2

### A preparation of O-(3,5-dimethoxy-4-(2-hydroxyethoxy)cinnamoyl)fumagillol

### Step 1: A preparation of O-(4-acetoxy-3,5-dimethoxycinnamoyl)fumagillol

The same procedure as described in the Step 1 of the Example 1 was repeated, but using a compound of the Chemical Formula 2 (1.0 g), 4-acetoxy-3,5-dimethoxycinnamic acid (2.36 g), thionylchloride (1.29 ml), toluene (20 ml), sodium hydride (850 mg) and dimethylformamide (20 ml), to give 1.36 g (72%) of the title compound as a white solid.

¹H-NMR (400 MHz, CDCl₃) δ: 7.59 (d, 1H, J=16 Hz), 6.77 (s, 2H), 6.44 (d, 1H, J=16 Hz), 5.71 (m, 1H), 5.21 (m, 1H), 3.86 (s, 3H), 3.71 (dd, 1H, J=11, 2.7 Hz), 3.45 (s, 3H), 3.0 (d, 1H, J=4.0 Hz), 2.62 (t, 1H, J=6.4 Hz), 2.57 (d, 1H, J=4.0 Hz), 2.36 (m, 1H), 2.34 (s, 3H), 2.20-2.04 (m, 4H), 1.89 (m, 1H), 1.74 (s, 3H), 1.65 (s, 3H), 1.23 (s, 3H), 1.10 (m, 1H).

### Step 2: A preparation of O-(3,5-dimethoxy-4-hydroxycinnamoyl)fumagillol

The same procedure as described in the step 2 of the Example 1 was repeated but using O-(4-acetoxy-3,5-dimethoxycinnamoyl)fumagillol (1.04 g), potassium carbonate (270 mg) and methanol (20 ml), to give 839 mg (88%) of the title compound as a white solid.

¹H-NMR (400 MHz, CDCl₃) δ: 7.58 (d, 1H, J=16 Hz), 6.78 (s, 2H), 6.37 (d, 1H, J=16 Hz), 5.72 (m, 2H), 5.21 (m, 1H), 3.93 (s, 6H), 3.71 (dd, 1H, J=11, 2.8 Hz), 3.45 (s, 3H), 3.00 (d, 1H, J=4 Hz), 2.62 (t, 1H, J=6.4 Hz), 2.57 (d, 1H, J=4 Hz), 2.36 (m, 1H), 2.20-2.04 (m, 4H), 1.88 (m, 1H), 1.74 (s, 3H), 1.66 (s, 3H), 1.23 (s, 3H), 1.11 (m, 1H).

### Step 3: A preparation of O-(3,5-dimethoxy-4-(2-hydroxyethoxy)cinnamoyl)fumagillol

The same procedure as described in the step 3 of Example 1 was repeated but using O-(3,5-dimethoxy-4-hydroxycinnamoyl)fumagillol (630 mg), potassium carbonate (1.07 g), 2-iodoethanol (0.4 ml) and dimethylformamide (20 ml), to give 610 mg (89%) of the title compound as a white solid.

¹H-NMR (400 MHz, CDCl₃) δ: 7.58 (d, 1H, J=16 Hz), 6.76 (s, 3H), 6.42 (d, 1H, J=16 Hz), 5.72 (m, 1H), 5.19 (m, 1H), 4.16 (m, 2H), 3.90 (s, 3H), 3.72 (m, 2H), 3.45 (s, 3H), 2.99 (d, 1H, J=4 Hz), 2.61 (t, 1H, J=6.4 Hz), 2.57 (d, 1H, J=4 Hz), 2.41 (m, 1H), 2.20-2.03 (m, 4H), 1.92 (m, 1H), 1.74 (s, 3H), 1.65 (s, 3H), 1.23 (s, 3H), 1.11 (m, 1H).

### Example 3

### A preparation of O-(4-(2-hydroxyethoxy)-3-methoxycinnamoyl)fumagillol

### Step 1: A preparation of O-(4-acetoxy-3-methoxycinnamoyl)fumagillol

The sane procedure as described in the step 1 of Example 1 was repeated but using a compound of the Chemical Formula 2 (1.0 g), 4-acetoxy-3-methoxycinnamic acid (2.09 g), thionylchloride (1.29 ml), toluene (20 ml), triethylamine (2.7 ml) and dichloromethane (20 ml), to give 1.0 g (56%) of the title compound as light yellow syrub.

¹H-NMR (400 MHz, CDCl₃) δ: 7.62 (d, 1H, J=16 Hz), 7.13-7.03 (m, 3H), 6.44 (d, 1H, J=16 Hz), 5.73 (m, 1H), 5.43 (m, 1H), 5.21 (m, 1H), 3.88 (s, 3H), 3.71 (dd, 1H, J=11.2, 2.8 Hz), 3.45 (s, 3H), 3.00 (d, 1H, J=4 Hz), 2.62 (t, 1H, J=6.3 Hz), 2.57 (d, 1H, J=4 Hz), 2.35 (m, 1H), 2.32 (s, 3H), 2.20-2.04 (m, 4H), 1.89 (m, 1H), 1.74 (s, 3H), 1.65 (s, 3H), 1.23 (s, 3H), 1.11 (m, 1H).

### Step 2: A preparation of O-(4-hydroxy-3-methoxycinnamoyl)fumagillol

The same procedure as described in the step 2 of Example 1 was repeated but using O-(4-acetoxy-3-methoxycinnamoyl)fumagillol (1.0 g), potassium carbonate (276 mg) and methanol (20 ml), to give 825 mg (90%) of the title compound as white solid.

¹H-NMR (400 MHz, CDCl₃) δ: 7.59 (d, 1H, J=16 Hz), 7.03 (m, 2H), 6.90 (d, 1H, J=7.9 Hz), 6.34 (d, 1H, J=16 Hz), 5.86 (s, 1H), 5.72 (m, 1H), 5.21 (m, 1H), 3.94 (s, 3H), 3.71 (dd, 1H, J=11.2, 2.8 Hz), 3.45 (s, 3H), 3.01 (d, 1H, J=4 Hz), 2.60 (t, 1H, J=6.3 Hz), 2.57 (d, 1H, J=4 Hz), 2.35 (m, 1H), 2.20-2.04 (m, 4H), 1.88 (m, 1H), 1.75 (s, 3H), 1.66 (s, 3H), 1.22 (s, 3H), 1.11 (m, 1H).

### Step 3: A preparation of O-(4-(2-hydroxyethoxy)-3-methoxycinnamoyl)fumagillol

The same procedure as described in the step 3 of Example 1 was repeated but using O-(4-hydroxy-3-methoxycinnamoyl)fumagillol (565 mg), potassium carbonate (1.02 g), 2-iodoethanol (0.39 ml) and dimethylformamide (20 mL), to give 380 mg (61%) of the title compound as white solid.

¹H-NMR (400 MHz, CDCl₃) δ: 7.61 (d, 1H, J=16 Hz), 7.06 (m, 2H), 6.89 (d, 1H, J=7.9 Hz), 6.38 (d, 1H, J=16 Hz), 5.72 (m, 1H), 5.21 (m, 1H), 4.16 (m, 2H), 3.98 (m, 2H), 3.91 (s, 3H), 3.71 (dd, 1H, J=11.2, 2.8 Hz), 3.45 (s, 3H), 3.01 (d, 1H, J=4 Hz), 2.62 (t, 1H, J=6.3 Hz), 2.57 (d, 1H, J=4 Hz), 2.38 (m, 1H), 2.25-2.04 (m, 4H), 1.88 (m, 1H), 1.75 (s, 3H), 1.66 (s, 3H), 1.66 (s, 3H), 1.23 (s, 3H), 1.11 (m, 1H).

### Example 4

### A preparation of O-(3-(2-hydroxyethoxy)-4-methoxycinnamoyl)fumagillol

### Step 1: A preparation of O-(3-acetoxy-4-methoxycinnamoyl)fumagillol

The same procedure as described in the step 1 of Example 1 was repeated but using a compound of the Chemical Formula 2 (1.0 g), 3-acetoxy-4-methoxycinnamic acid (2.09 g), thionylchloride (1.29 ml), toluene (20 ml), triethylamine (2.7 ml) and dichloromethane (20 ml), to give 1.01 g (59%) of the title compound as white solid.

¹H-NMR (400 MHz, CDCl₃) δ: 7.58 (d, 1H, J=16 Hz), 7.73 (m, 1H), 7.23 (m, 1H), 6.96 (d, 1H, J=8.5 Hz), 6.34 (d, 1H, J=16 Hz), 5.72 (m, 1H), 5.21 (m, 1H), 3.86 (s, 3H), 3.70 (dd, 1H, J=11, 2.7 Hz), 3.45 (s, 3H), 3.04 (d, 1H, J=4 Hz), 2.61 (t, 1H, J=6.3 Hz), 2.56 (d, 1H, J=4 Hz), 2.35 (m, 1H), 2.19-2.01 (m, 4H), 1.88 (m, 1H), 1.74 (s, 3H), 1.66 (s, 3H), 1.23 (s, 3H), 1.12 (m, 1H).

### Step 2: A preparation of O-(3-hydroxy-4-methoxycinnamoyl)fumagillol

The same procedure as described in the step 2 of Example 1 was repeated but using O-(3-acetoxy-4-methoxycinnamoyl)fumagillol (550 mg), potassium carbonate (156 mg) and methanol (10 ml), to give 450 mg (87%) of the title compound as white solid.

¹H-NMR (400 MHz, CDCl₃) δ: 7.58 (d, 1H, J=16 Hz), 7.12 (m, 1H), 7.04 (m, 1H), 6.84 (d, 1H, J=8.5 Hz), 6.34 (d, 1H, J=16 Hz), 5.74 (s, 1H), 5.61 (s, 1H), 5.22 (m, 1H), 3.92 (s, 3H), 3.70 (m, 1H), 3.45 (s, 3H), 3.00 (d, 1H, J=4 Hz), 2.62 (t, 1H, J=6.3 Hz), 2.57 (d, 1H, 4 Hz), 2.34 (m, 1H), 2.19-2.01 (m, 4H), 1.90 (m, 1H), 1.84 (s, 3H), 1.74 (s, 3H), 1.23 (s, 3H), 1.12 (m, 1H).

### Step 3: A preparation of O-(3-(2-hydroxyethoxy)-4-methoxycinnamoyl)fumagillol

The same procedure as described in the step 3 of Example 1 was repeated but using O-(3-hydroxy-4-methoxycinnamoyl)fumagillol (720 mg), potassium carbonate (1.34 g), 2-iodoethanol (0.51 mL) and dimethylformamide (20 ml), to give 400 mg (50%) of the title compound as white solid.

¹H-NMR (400 MHz, CDCl₃) δ: 7.59 (d, 1H, J=16 Hz), 7.12 (m, 2H), 6.88 (d, 1H, J=8.5 Hz), 6.37 (d, 1H, J=16 Hz), 5.73 (m, 1H), 5.22 (m, 1H), 4.18 (m, 2H), 3.97 (m, 2H), 3.90 (s, 3H), 3.71 (m, 1H), 3.45 (s, 3H), 3.00 (d, 1H, J=4 Hz), 2.62 (t, 1H, J=6.3 Hz), 2.57 (d, 1H, J=4 Hz), 2.35 (m, 1H), 2.20-2.03 (m, 4H), 1.90 (m, 1H), 1.84 (s, 3H), 1.74 (s, 3H), 1.23 (s, 3H), 1.12 (m, 1H).

### Example 5

### A preparation of O-(4-(2-hydroxyethylamino)cinnamoyl)fumagillol

### Step 1: A preparation of O-(4-acetaminocinnamoyl)fumagillol

The same procedure as described in the step 1 of Example 1 was repeated but using a compound of the Chemical Formula 2 (560 mg), 4-acetaminocinnamic acid (1.02 g), thionylchloride (0.72 ml), toluene (30 ml), sodium hydride (478 mg) and dimethylformamide (10 ml), to give 200 mg (21%) of the title compound as white solid.

¹H-NMR (400 MHz, CDCl₃) δ: 7.66 (d, 1H, J=16 Hz), 7.54 (m, 2H), 7.13 (m, 2H), 6.44 (d, 1H, J=16 Hz), 5.75 (m, 1H), 5.20 (m, 1H), 3.70 (m, 1H), 3.45 (s, 3H), 3.00 (d, 1H, J=4 Hz), 2.60 (t, 1H, J=6.3 Hz), 2.56 (d, 1H, J=4 Hz), 2.35 (m, 1H), 2.20-2.04 (m, 7H), 1.89 (m, 1H), 1.74 (s, 3H), 1.64 (s, 3H), 1.20 (s, 3H), 1.11 (m, 1H).

### Step 2: A preparation of O-(4-aminocinnamoyl)fumagillol

The same procedure as described in the step 2 of Example 1 was repeated but using O-(4-acetaminocinnamoyl)fumagillol (200 mg), potassium carbonate (58 mg) and ethanol (20 mL), to give 100 mg (54%) of the title compound as white solid.

¹H-NMR (400 MHz, CDCl₃) δ: 7.55 (d, 1H, J=16 Hz), 7.34 (m, 2H), 6.74 (m, 2H), 6.29 (d, 1H, J=16 Hz), 5.73 (m, 1H), 5.19 (m, 1H), 3.69 (m, 1H), 3.45 (s, 3H), 3.00 (d, 1H, J=4 Hz), 2.64 (t, 1H, J=6.3 Hz), 2.56 (d, 1H, J=4 Hz), 2.36 (m, 1H), 2.17-2.01 (m, 4H), 1.88 (m, 1H), 1.74 (s, 3H), 1.65 (s, 3H), 1.24 (s, 3H), 1.11 (m, 1H).

### Step 3: A preparation of O-(4-(2-hydroxyethylamino)cinnamoyl)fumagillol

The same procedure as described in the step 3 of Example 1 was repeated but using O-(4-aminocinnamoyl)fumagillol (100 mg), sodium carbonate (149 mg), 2-iodoethanol (73 μl) and dimethylformamide (5 ml), to give 10 mg (9%) of the title compound as yellow solid.

¹H-NMR (400 MHz, CDCl₃) δ: 7.57 (d, 1H, J=16 Hz), 7.34 (m, 2H), 6.74 (m, 2H), 6.44 (d, 1H, J=16 Hz), 5.74 (m, 1H), 5.21 (m, 1H), 3.87 (m, 2H), 3.70 (m, 1H), 3.47 (s, 3H), 3.36 (m, 2H), 3.00 (d, 1H, J=4 Hz), 2.63 (m, 1H), 2.57 (d, 1H, J=4 Hz), 2.40 (m, 1H), 2.20-1.88 (m, 5H), 1.74 (s, 3H), 1.66 (s, 3H), 1.25 (s, 3H), 1.11 (m, 1H).

### Example 6

### A preparation of O-(3-(2-hydroxyethylamino)cinnamoyl)fumagillol

### Step 1: A preparation of O-(3-acetaminocinnamoyl)fumagillol

The same procedure as described in the step 1 of Example 1 was repeated but using a compound of the Chemical Formula 2 (1.0 g), 3-acetaminocinnamic acid (1.8 g), toluene (30 mL), thionylchloride (1.29 ml), sodium hydride (850 mg) and dimethylformamide (20 ml), to give 300 mg (18%) of the title compound as colorless syrup.

¹H-NMR (400 MHz, CDCl₃) δ: 7.65 (d, 1H, J=16 Hz), 7.20 (m, 1H), 6.92 (m, 1H), 6.88 (s, 1H), 6.74 (m, 1H), 6.44 (d, 1H, J=16 Hz), 5.74 (m, 1H), 5.21 (m, 1H), 3.72 (m, 1H), 3.47 (s, 3H), 3.00 (d, 1H, J=4 Hz), 2.63 (t, 1H, J=6.4 Hz), 2.56 (d, 1H, J=4 Hz), 2.38 (m, 1H), 2.19-1.88 (m, 8H), 1.74 (s, 3H), 1.66 (s, 3H), 1.25 (s, 3H), 1.11 (m, 1H).

### Step 2: A preparation of O-(3-aminocinnamoyl)fumagillol

The same procedure as described in the step 2 of Example 1 was repeated but using O-(3-acetaminocinnamoyl)fumagillol (200 mg), potassium carbonate (58 mg) and ethanol (20 ml), to give 120 mg (65%) of the title compound as white solid.

¹H-NMR (400 MHz, CDCl₃) δ: 7.66 (d, 1H, J=16 Hz), 7.20 (m, 1H), 6.93 (m, 1H), 6.88 (s, 1H), 6.75 (m, 1H), 6.45 (d, 1H, J=16 Hz), 5.75 (m, 1H), 5.21 (m, 1H), 3.72 (m, 1H), 3.47 (s, 3H), 3.00 (d, 1H, J=4 Hz), 2.63 (t, 1H, J=6.3 Hz), 2.56 (d, 1H, J=4 Hz), 2.38 (m, 1H), 2.19-1.88 (m, 5H), 1.74 (s, 3H), 1.66 (s, 3H), 1.25 (s, 3H), 1.12 (m, 1H)

### Step 3: A preparation of O-(3-(2-hydroxyethylamino)cinnamoyl)fumagillol

The same procedure as described in the step 3 of Example 1 was repeated but using O-(3-aminocinnamoyl)fumagillol (100 mg), sodium carbonate (149 mg), 2-iodoethanol (73 μl) and dimethylformamide (5 ml), to give 15 mg (15%) of the title compound as white solid.

¹H-NMR (400 MHz, CDCl₃) δ: 7.57 (d, 1H, J=16 Hz), 7.20 (t, 1H, J=7.8 Hz), 6.92 (d, 1H, J=7.6 Hz), 6.88 (s, 1H), 6.74 (m, 1H), 6.44 (d, 1H, J=16 Hz), 5.74 (m, 1H), 5.21 (m, 1H), 3.87 (t, 2H, J=5.1 Hz), 3.71 (dd, 1H, J=11, 2.8 Hz), 3.47 (s, 3H), 3.36 (t, 2H, J=5.1 Hz), 3.00 (d, 1H, J=4 Hz), 2.63 (m, 1H), 2.57 (d, 1H, J=4 Hz), 2.40 (m, 1H), 2.20-1.89 (m, 5H), 1.75 (s, 3H), 1.67 (s, 3H), 1.23 (s, 3H), 1.11 (m, 1H).

### Example 7

### A preparation of O-(4-chloro-3-(2-hydroxyethylamino)cinnamoyl)fumagillol

### Step 1: A preparation of O-(4-chloro-3-acetaminocinnamoyl)fumagillol

The same procedure as described in the step 1 of Example 1 was repeated but using a compound of the Chemical Formula 2 (1.0 g), 4-chloro-3-acetaminocinnamic acid (2.02 g), thionylchloride (1.29 ml), toluene (30 ml), sodium hydride (850 mg) and dimethylformamide (20 ml), to give 200 mg (11%) of the title compound as colorless syrup.

¹H-NMR (400 MHz, CDCl₃) δ: 7.65 (d, 1H, J=16 Hz), 7.20 (m, 1H), 6.88 (s, 1H), 6.74 (m, 1H), 6.44 (d, 1H, J=16 Hz), 5.74 (m, 1H), 5.21 (m, 1H), 3.72 (m, 1H), 3.47 (s, 3H), 3.00 (d, 1H, J=4 Hz), 2.63 (t, 1H J=6.3 Hz), 2.56 (d, 1H, J=4 Hz), 2.38 (m, 1H), 2.19-1.89 (m, 8H), 1.74 (s, 3H), 1.65 (s, 3H), 1.25 (s, 3H), 1.11 (m, 1H).

### Step 2: A preparation of O-(4-chloro-3-aminocinnamoyl)fumagillol

The same procedure as described in the step 2 of Example 1 was repeated but using O-(3-acetamino-4-chlorocinnamoyl)fumagillol (200 mg), potassium carbonate (55 mg) and ethanol (20 ml), to give 100 mg (54%) of the title compound as white solid.

¹H-NMR (400 MHz, CDCl₃) δ: 7.65 (d, 1H, J=16 Hz), 7.20 (m, 1H), 6.88 (s, 1H), 6.74 (m, 1H), 6.44 (d, 1H, J=16 Hz), 5.74 (m, 1H), 5.21 (m, 1H), 3.72 (m, 1H), 3.47 (s, 3H), 3.01 (d, 1H, J=4 Hz), 2.63 (t, 1H J=6.3 Hz), 2.56 (d, 1H, J=4 Hz), 2.38 (m, 1H), 2.19-1.89 (m, 5H), 1.74 (s, 3H), 1.65 (s, 3H), 1.25 (s, 3H), 1.12 (m, 1H).

### Step 3: A preparation of O-(4-chloro-3-(2-hydroxyethylamino)cinnamoyl)fumagillol

The same procedure as described in the step 3 of Example 1 was repeated but using O-(3-amino-4-chlorocinnamoyl)fumagillol (100 mg), sodium carbonate (140 mg), 2-iodoethanol (69 μl) and dimethylformamide (5 ml), to give 60 mg (54%) of the title compound as white solid.

¹H-NMR (400 MHz, CDCl₃) δ: 7.65 (d, 1H, J=16 Hz), 7.20 (m, 1H), 6.88 (s, 1H), 6.74 (m, 1H), 6.44 (d, 1H, J=16 Hz), 5.74 (m, 1H), 5.21 (m, 1H), 3.87 (m, 2H), 3.72 (m, 1H), 3.47 (s, 3H), 3.36 (m, 2H), 3.01 (d, 1H, J=4 Hz), 2.63 (t, 1H, J=6.4 Hz), 2.56 (d, 1H, J=4 Hz), 2.38 (m, 1H), 2.19-1.88 (m, 5H), 1.74 (s, 3H), 1.65 (s, 3H), 1.25 (s, 3H), 1.11 (m, 1H).

### Example 8

### A preparation of O-(4-(4-hydroxymethylphenoxy)cinnamoyl)fumagillol

### Step 1: A preparation of O-(4-(4-acetoxymethylphenoxy)cinnamoyl)fumagillol

The same procedure as described in the step 1 of Example 1 was repeated but using a compound of the Chemical Formula 2 (827 mg), 4-(4-acetoxymethylphenoxy)cinnamic acid (2.29 g), thionylchloride (1.07 ml), toluene (40 ml), sodium hydride (703 mg) and dimethylformamide (20 ml), to give 1.14 g (67%) of the title compound as colorless syrup.

¹H-NMR (400 MHz, CDCl₃) δ: 7.63 (d, 1H, J=16 Hz), 7.48 (d, 2H, J=8.6 Hz), 7.36 (d, 2H, J=8.4 Hz), 7.03 (d, 2H, J=8.4 Hz), 6.98 (d, 2H, J=8.6 Hz), 6.40 (d, 1H, J=16 Hz), 5.74 (s, 1H), 5.21 (m, 1H), 5.08 (s, 2H), 3.70 (d, 1H, J=11, 2.7 Hz), 3.45 (s, 3H), 3.01 (d, 1H, J=4 Hz), 2.61 (t, 1H, J=6.3 Hz), 2.57 (d, 1H, J=4 Hz), 2.36 (m, 1H), 2.19-1.88 (m, 8H), 1.74 (s, 3H), 1.65 (s, 3H), 1.23 (s, 3H), 1.12 (m, 1H).

### Step 2: A preparation of O-(4-(4-hydroxymethylphenoxy)cinnamoyl)fumagillol

The same procedure as described in the step 2 of Example 1 was repeated but using O-(4-(4-acetoxymethylphenoxy)cinnamoyl)fumagillol (1.14 g), cesium carbonate (644 mg) and methanol (20 ml), to give 811 mg (77%) of the title compound as white solid.

¹H-NMR (400 MHz, CDCl₃) δ: 7.63 (d, 1H, J=16 Hz), 7.48 (d, 2H, J=8.6 Hz), 7.36 (d, 2H, J=8.4 Hz), 7.03 (d, 2H, J=8.4 Hz), 6.98 (d, 2H, J=8.6 Hz), 6.40 (d, 1H, J=16 Hz), 5.74 (m, 1H), 5.21 (m, 1H), 4.69 (s, 2H), 3.70 (dd, 1H, J=11, 2.7 Hz), 3.45 (s, 3H), 3.01 (d, 1H, J=4 Hz), 2.61 (t, 1H, J=6.3 Hz), 2.57 (d, 1H, J=4 Hz), 2.36 (m, 1H), 2.19-1.88 (m, 5H), 1.74 (s, 3H), 1.65 (s, 3H), 1.23 (s, 3H), 1.11 (m, 1H).

### Example 9

### A preparation of O-(3,5-dimethoxy-4-(4-hydroxymethylphenoxy)cinnamoyl)fumagillol

### Step 1: A preparation of O-(4-(4-acetoxymethylphenoxy)-3,5-dimethoxycinnamoyl)fumagillol

The same procedure as described in the step 1 of Example 1 was repeated but using a compound of the Chemical Formula 2 (1.0 g), 4-(4-acetoxymethylphenoxy)-3,5-dimethoxycinnamic acid (3.3 g), thionylchloride (1.29 ml), toluene (40 ml), sodium hydride (850 mg) and dimethylformamide (20 ml), to give 1.2 g (53%) of the title compound as white solid.

¹H-NMR (400 MHz, CDCl₃) δ: 7.64 (d, 1H, J=16 Hz), 7.36 (d, 2H, J=8.4 Hz), 7.03 (d, 2H, J=8.4 Hz), 6.76 (s, 2H), 6.40 (d, 1H, J=16 Hz), 5.74 (s, 1H), 5.21 (m, 1H), 5.08 (s, 2H), 3.98 (s, 6H), 3.70 (dd, 1H, J=11, 2.7 Hz), 3.45 (s, 3H), 3.01 (d, 1H, J=4 Hz), 2.61 (t, 1H, J=6.3 Hz), 2.57 (d, 1H, J=4 Hz), 2.36 (m, 1H), 2.19-1.88 (m, 8H), 1.74 (s, 3H), 1.65 (s, 3H), 1.23 (s, 3H), 1.11 (m, 1H).

### Step 2: A preparation of O-(3,5-dimethoxy-4-(4-hydroxymethylphenoxy)cinnamoyl)fumagillol

The same procedure as described in the step 2 of Example 1 was repeated but using O-(4-(4-acetoxymethylphenoxy)-3,5-dimethoxycinnamoyl)fumagillol (1.2 g), cesium carbonate (614 mg) and methanol (20 ml), to give 1.0 g (89%) of the title compound as white solid.

¹H-NMR (400 MHz, CDCl₃) δ: 7.64 (d, 1H, J=16 Hz), 7.36 (d, 2H, J=8.4 Hz), 7.03 (d, 2H, J=8.4 Hz), 6.76 (s, 2H), 6.40 (d, 1H, J=16 Hz), 5.74 (s, 1H), 5.21 (m, 1H), 5.08 (s, 2H), 3.98 (s, 6H), 3.70 (dd, 1H, J=11, 2.7 Hz), 3.45 (s, 3H), 3.01 (d, 1H, J=4 Hz), 2.61 (t, 1H, J=6.3 Hz), 2.57 (d, 1H, J=4 Hz), 2.36 (m, 1H), 2.19-1.88 (m, 5H), 1.74 (s, 3H), 1.65 (s, 3H), 1.23 (s, 3H), 1.11 (m, 1H).

### Example 10

### A preparation of O-(4-(4-hydroxymethylphenoxy)-3-methoxycinnamoyl)fumagillol

### Step 1: A preparation of O-(4-(4-acetoxymethylphenoxy)-3-methoxycinnamoyl)fumagillol

The same procedure as described in the step 1 of Example 1 was repeated but using a compound of the Chemical Formula 2 (1.0 g), 4-(4-acetoxymethylphenoxy)-3-methoxycinnamic acid (3.03 g), thionylchloride (1.29 ml), toluene (30 ml), sodium hydride (850 mg) and dimethylformamide (20 ml), to give 1.05 g (49%) of the title compound as white solid.

¹H-NMR (400 MHz, CDCl₃) δ: 7.63 (d, 1H, J=16 Hz), 7.48 (d, 1H, J=8.6 Hz), 7.36 (d, 2H, J=8.4 Hz), 7.03 (d, 2H, J=8.4 Hz), 6.98 (d, 1H, J=8.6 Hz), 6.76 (s, 1H), 6.40 (d, 1H, J=16 Hz), 5.74 (s, 1H), 5.21 (m, 1H), 5.08 (s, 2H), 3.94 (s, 3H), 3.70 (dd, 11, J=11, 2.7 Hz), 3.45 (s, 3H), 3.01 (d, 1H, J=4 Hz), 2.61 (t, 1H, J=6.3 Hz), 2.57 (d, 1H, J=4 Hz), 2.36 (m, 1H), 2.19-1.89 (m, 8H), 1.74 (s, 3H), 1.65 (s, 3H), 1.24 (s, 3H), 1.11 (m, 1H).

### Step 2: A preparation of O-(4-(4-hydroxymethylphenoxy)-3-methoxycinnamoyl)fumagillol

The same procedure as described in the step 2 of Example 1 was repeated but using O-4-(4-acetoxymethylphenoxy)-3-methoxycinnamoyl)fumagillol (1 g), cesium carbonate (537 mg) and methanol (20 ml), to give 800 mg (86%) of the title compound as white solid.

¹H-NMR (400 MHz, CDCl₃) δ: 7.63 (d, 1H, J=16 Hz), 7.48 (d, 1H, J=8.6 Hz), 7.36 (d, 2H, J=8.4 Hz), 7.03 (d, 2H, J=8.4 Hz), 6.98 (d, 1H, J=8.6 Hz), 6.76 (s, 1H), 6.40 (d, 1H, J=16 Hz), 5.74 (s, 1H), 5.21 (m, 1H), 5.08 (s, 2H), 3.94 (s, 3H), 3.70 (dd, 1H, J=11, 2.7 Hz), 3.45 (s, 3H), 3.01 (d, 1H, J=4 Hz), 2.61 (t, 1H, J=6.3 Hz), 2.57 (d, 1H, J=4 Hz), 2.36 (m, 1H), 2.20-1.88 (m, 5H), 1.74 (s, 3H), 1.65 (s, 3H), 1.24 (s, 3H), 1.11 (m, 1H).

### Example 11

### A preparation of O-(3-(4-hydroxymethylphenoxy)-4-methoxycinnamoyl)fumagillol

### Step 1: A preparation of O-(3-(4-acetoxymethylphenoxy)-4-methoxycinnamoyl)fumagillol

The same procedure as described in the step 1 of Example 1 was repeated but using a compound of the Chemical Formula 2 (1.0 g), 3-(4-acetoxymethylphenoxy)-4-methoxycinnamic acid (3.03 g), thionylchloride (1.29 ml), toluene (30 ml), sodium hydride (850 mg) and dimethyl formamide (20 ml), to give 950 mg (44%) of the title compound as white solid.

¹H-NMR (400 MHz, CDCl₃) δ: 7.63 (d, 1H, J=16 Hz), 7.48 (d, 1H, J=8.6 Hz), 7.36 (d, 2H, J=8.4 Hz), 7.12 (m, 1H), 7.03 (m, 3H), 6.41 (d, 1H, J=16 Hz), 5.74 (s, 1H), 5.21 (m, 1H), 5.08 (s, 2H), 3.94 (s, 3H), 3.70 (m, 1H), 3.45 (s, 3H), 3.00 (m, 1H), 2.61 (t, 1H, J=6.3 Hz), 2.57 (m, 1H), 2.36 (m, 1H), 2.20-1.88 (m, 8H), 1.74 (s, 3H), 1.65 (s, 3H), 1.25 (s, 3H), 1.12 (m, 1H).

### Step 2: A preparation of O-(3-(4-hydroxymethylphenoxy)-4-methoxycinnamoyl)fumagillol

The same procedure as described in the step 2 of Example 1 was repeated but using O-(3-(4-acetoxymethylphenoxy)-4-methoxycinnamoyl)fumagillol (950 mg), cesium carbonate (510 mg) and methanol (20 ml), to give 760 mg (86%) of the title compound as white solid.

¹H-NMR (400 MHz, CDCl₃) δ: 7.63 (d, 1H, J=16 Hz), 7.48 (d, 1H, J=8.6 Hz), 7.36 (d, 2H, J=8.4 Hz), 7.12 (m, 1H), 7.03 (m, 3H), 6.41 (d, 1H, J=16 Hz), 5.74 (s, 1H), 5.21 (m, 1H), 5.08 (s, 2H), 3.94 (s, 3H), 3.70 (m, 1H), 3.45 (s, 3H), 3.00 (m, 1H), 2.61 (t, 1H, J=6.3 Hz), 2.57 (m, 1H), 2.36 (m, 1H), 2.20-1.88 (m, 5H), 1.74 (s, 3H), 1.65 (s, 3H), 1.25 (s, 3H), 1.12 (m, 1H).

### Example 12

### A preparation of O-(4-(2-hydroxyethoxyethoxy)cinnamoyl)fumagillol

The same procedure as described in the step 3 of Example 1 was repeated but using O-(4-hydroxycinnamoyl)fumagillol (500 mg), potassium carbonate (968 mg), 2-(2-chloroethoxy)ethanol (0.49 ml) and dimethylformamide (20 ml), to give 300 mg (50%) of the title compound as white solid.

¹H-NMR (400 MHz, CDCl₃) δ: 7.61 (d, 1H, J=16 Hz), 7.19 (m, 2H), 6.72 (m, 2H), 6.35 (d, 1H, J=16 Hz), 5.73 (m, 1H), 5.21 (m, 1H), 4.11 (m, 2H), 3.79 (m, 2H), 3.69-3.70 (m, 3H), 3.56 (m, 2H), 3.45 (s, 3H), 2.99 (d, 1H, J=4.4 Hz), 2.60 (t, 1H, J=6.4 Hz), 2.56 (d, 1H, J=4.4 Hz), 2.35 (m, 1H), 2.19-2.00 (m, 4H), 1.90 (m, 1H), 1.74 (s, 3H), 1.65 (s, 3H), 1.22 (s, 3H), 1.08 (m, 1H).

### Example 13

### A preparation of O-(3,5-dimethoxy-4-(2-hydroxyethoxyethoxy)cinnamoyl)fumagillol

The same procedure as described in the step 3 of Example 1 was repeated but using O-(3,5-dimethoxy-4-hydroxycinnamoyl)fumagillol (500 mg), potassium carbonate (849 mg), 2-(2-chloroethoxy)ethanol (0.43 ml) and dimethylformamide (20 ml), to give 325 mg (55%) of the title compound as white solid.

¹H-NMR (400 MHz, CDCl₃) δ: 7.61 (d, 1H, J=16 Hz), 6.35 (d, 1H, J=16 Hz), 6.26 (s, 2H), 5.73 (m, 1H), 5.21 (m, 1H), 4.11 (m, 2H), 4.11 (m, 2H), 3.79 (m, 2H), 3.73 (s, 6H), 3.69-3.70 (m, 3H), 3.56 (m, 2H), 3.45 (s, 3H), 2.99 (d, 1H, J=4.4 Hz), 2.60 (t, 1H, J=6.4 Hz), 2.56 (d, 1H, J=4.4 Hz), 2.35 (m, 1H), 2.19-2.00 (m, 4H), 1.90 (m, 1H), 1.74 (s, 3H), 1.65 (s, 3H), 1.22 (s, 3H), 1.08 (m, 1H).

### Example 14

### A preparation of O-(4-(2-hydroxyethoxyethoxy)-3-methoxycinnamoyl)fumagillol

The same procedure as described in the step 3 of Example 1 was repeated but using O-(4-hydroxy-3-methoxycinnamoyl)fumagillol (500 mg), potassium carbonate (904 mg), 2-(2-chloroethoxy)ethanol (0.46 ml) and dimethylformamide (20 ml), to give 290 mg (49%) of the title compound as white solid.

¹H-NMR (400 MHz, CDCl₃) δ: 7.61 (d, 11H, J=16 Hz), 6.75 (m, 1H), 6.70 (s, 1H), 6.61 (m, 1H), 6.35 (d, 1H, J=16 Hz), 5.73 (m, 1H), 5.21 (m, 1H), 4.11 (m, 2H), 3.79 (m, 2H), 3.73 (s, 3H), 3.69-3.70 (m, 3H), 3.56 (m, 2H), 3.45 (s, 3H), 2.99 (d, 1H, J=4.4 Hz), 2.60 (t, 1H, J=6.4 Hz), 2.56 (d, 1H, J=4.4 Hz), 2.35 (m, 1H), 2.19-2.00 (m, 4H), 1.90 (m, 1H), 1.74 (s, 3H), 1.65 (s, 3H), 1.22 (s, 3H), 1.08 (m, 1H).

### Example 15

### A preparation of O-(3-(2-hydroxyethoxyethoxy)-4-methoxycinnamoyl)fumagillol

The same procedure as described in the step 3 of Example 1 was repeated but using O-(3-hydroxy-4-methoxycinnamoyl)fumagillol (500 mg), potassium carbonate (904 mg), 2-(2-chloroethoxy)ethanol (0.46 ml) and dimethylformamide (20 ml), to give 250 mg (42%) of the title compound as white solid.

¹H-NMR (400 MHz, CDCl₃) δ: 7.61 (d, 1H, J=16 Hz), 6.75 (m, 1H), 6.70 (s, 1H), 6.61 (m, 1H), 6.35 (d, 1H, J=16 Hz), 5.73 (m, 1H), 5.21 (m, 1H), 4.11 (m, 2H), 3.79 (m, 2H), 3.73 (s, 3H), 3.69-3.70 (m, 3H), 3.56 (m, 2H), 3.45 (s, 3H), 2.99 (d, 1H, J=4.4 Hz), 2.60 (t, 1H, J=6.4 Hz), 2.56 (d, 1H, J=4.4 Hz), 2.35 (m, 1H), 2.19-2.00 (m, 4H), 1.90 (m, 1H), 1.74 (s, 3H), 1.65 (s, 3H), 1.22 (s, 3H), 1.08 (m, 1H).

### Example 16

### A preparation of O-(4-2-hydroxyethoxyethylamino)cinnamoyl)fumagillol

The same procedure as described in the step 3 of Example 1 was repeated but using O-(4-aminocinnamoyl)fumagillol (500 mg), potassium carbonate (970 mg), 2-(2-chloroethoxy)ethanol (0.49 ml) and dimethylformamide (20 ml), to give 100 mg (17%) of the title compound as white solid.

¹H-NMR (400 MHz, CDCl₃) δ: 7.61 (d, 1H, J=16 Hz), 7.08 (m, 2H), 6.38 (m, 2H), 6.35 (d, 1H, J=16 Hz), 5.73 (m, 1H), 5.21 (m, 1H), 3.69-3.70 (m, 3H), 3.60 (m, 2H), 3.56 (m, 2H), 3.45 (s, 3H), 3.23 (m, 2H), 2.99 (d, 1H, J=4.4 Hz), 2.60 (t, 1H, J=6.4 Hz), 2.56 (d, 1H, J=4.4 Hz), 2.35 (m, 1H), 2.19-2.00 (m, 4H), 1.90 (m, 1H), 1.74 (s, 3H), 1.65 (s, 3H), 1.22 (s, 3H), 1.08 (m, 1H).

### Example 17

### A preparation of O-(3-(2-hydroxyethoxyethylamino)cinnamoyl)fumagillol)

The same procedure as described in the step 3 of Example 1 was repeated but using O-(3-aminocinnamoyl)fumagillol (500 mg), potassium carbonate (970 mg), 2-(2-chloroethoxy)ethanol (0.49 ml) and dimethylformamide (20 ml), to give 95 mg (16%) of the title compound as white solid.

¹H-NMR (400 MHz, CDCl₃) δ 7.61 (d, 1H, J=16 Hz), 6.99 (m, 1H), 6.62 (m, 1H), 6.47 (m, 1H), 6.35 (d, 1H, J=16 Hz), 6.31 (m, 1H), 5.73 (m, 1H), 5.21 (m, 1H), 3.69-3.70 (m, 3H), 3.60 (m, 2H), 3.56 (m, 2H), 3.45 (s, 3H), 3.23 (m, 2H), 2.99 (d, 1H, J=4.4 Hz), 2.60 (t, 1H, J=6.4 Hz), 2.56 (d, 1H, J=4.4 Hz), 2.35 (m, 1H), 2.19-2.00 (m, 4H), 1.90 (m, 1H), 1.74 (s, 3H), 1.65 (s, 3H), 1.22 (s, 3H), 1.08 (m, 1H).

### Example 18

### A preparation of O-(4-chloro-3-(2-hydroxyethoxyethylamino)cinnamoyl)fumagillol)

The same procedure as described in the step 3 of Example 1 was repeated but using O-(3-amino-4-chlorocinnamoyl)fumagillol (300 mg), potassium carbonate (538 mg), 2-(2-chloroethoxy)ethanol (0.27 ml) and dimethylformamide (20 ml), to give 55 mg (15%) of the title compound as white solid.

¹H-NMR (400 MHz, CDCl₃) δ: 7.61 (d, 1H, J=16 Hz), 7.00 (s, 1H), 6.56 (m, 1H), 6.41 (s, 1H), 6.35 (d, 1H, J=16 Hz), 5.71 (m, 1H), 5.23 (m, 1H), 3.70-3.72 (m, 3H), 3.61 (m, 2H), 3.56 (m, 2H), 3.45 (s, 3H), 3.23 (m, 2H), 2.99 (d, 1H, J=4.4 Hz), 2.59 (t, 1H, J=6.4 Hz), 2.55 (d, 1H, J=4.4 Hz), 2.35 (m, 1H), 2.18-2.01 (m, 4H), 1.90 (m, 1H), 1.74 (s, 3H), 1.65 (s, 3H), 1.22 (s, 3H), 1.08 (m, 1H).

### Example 19

### A preparation of O-(4-(3-hydroxypropoxy)cinnamoyl)fumagillol)

The same procedure as described in the step 3 of Example 1 was repeated but using O-(4-hydroxycinnamoyl)fumagillol (200 mg), potassium carbonate (387 mg), 3-chloropropanol (0.16 ml) and dimethylformamide (10 ml), to give 175 mg (77%) of the title compound as white solid.

¹H-NMR (400 MHz, CDCl₃) δ: 7.64 (d, 1H, J=16 Hz), 7.19 (m, 2H), 6.72 (m, 2H), 6.36 (d, 1H, J=16 Hz), 5.72 (m, 1H), 5.21 (m, 1H), 3.94 (m, 2H), 3.69 (dd, 1H, J=11, 2.7 Hz), 3.53 (m, 2H), 3.46 (s, 3H), 2.99 (d, 1H, J=4.4 Hz), 2.59 (t, 1H, J=6.4 Hz), 2.56 (d, 1H, J=4.4 Hz), 2.35 (m, 1H), 2.19-2.00 (m, 4H), 1.90 (m, 3H), 1.74 (s, 3H), 1.65 (s, 3H), 1.21 (s, 3H), 1.08 (m, 1H).

### Example 20

### A preparation of O-(3-cyano-4-(3-hydroxypropoxy)cinnamoyl)fumagillol

The same procedure as described in the step 3 of Example 1 was repeated but using O-(3-cyano-4-hydroxycinnamoyl)fumagillol (300 mg), potassium carbonate (509 mg), 3-chloropropanol (0.21 ml) and dimethylformamide (10 ml), to give 250 mg (74%) of the title compound as white solid.

¹H-NMR (400 MHz, CDCl₃) δ: 7.62 (d, 1H, J=16 Hz), 7.23 (m, 2H), 6.35 (d, 1H, J=16 Hz), 6.26 (s, 1H), 5.73 (m, 1H), 5.21 (m, 1H), 3.94 (m, 2H), 3.69 (dd, 1H, J=11, 2.7 Hz), 3.53 (m, 2H), 3.46 (s, 3H), 2.99 (d, 1H, J=4.4 Hz), 2.61 (t, 1H, J=6.4 Hz), 2.55 (d, 1H, J=4.4 Hz), 2.35 (m, 1H), 2.19-2.02 (m, 4H), 1.90-1.88 (m, 3H), 1.74 (s, 3H), 1.67 (s, 3H), 1.22 (s, 3H), 1.07 (m, 1H).

### Example 21

### A preparation of O-(4-(4-hydroxybutoxy)cinnamoyl)fumagillol

The same procedure as described in the step 3 of Example 1 was repeated but using O-(4-hydroxycinnamoyl)fumagillol (200 mg), potassium carbonate (387 mg), 4-chlorobutanol (0.19 ml) and dimethylformamide (15 ml), to give 150 mg (64%) of the title compound as white solid.

¹H-NMR (400 MHz, CDCl₃) δ: 7.62 (d, 1H, J=16 Hz), 7.19 (m, 2H), 6.73 (m, 2H), 6.34 (d, 11H, J=16 Hz), 5.75 (m, 1H), 5.22 (m, 1H), 3.94 (m, 2H), 3.70 (dd, 1H, J=11, 2.7 Hz), 3.55 (m, 2H), 3.45 (s, 3H), 2.97 (d, 1H, J=4.4 Hz), 2.61 (t, 1H, J=6.4 Hz), 2.54 (d, 1H, J=4.4 Hz), 2.35 (m, 1H), 2.20-2.03 (m, 4H), 1.91 (m, 1H), 1.75 (s, 3H), 1.70 (m, 2H), 1.64 (s, 3H), 1.48 (m, 2H), 1.22 (s, 3H), 1.07 (m, 1H).

### Example 22

### A preparation of O-(3-methyl-4-(4-hydroxybutoxy)cinnamoyl)fumagillol

The same procedure as described in the step 3 of Example 1 was repeated but using O-(3-methyl-4-hydroxycinnamoyl)fumagillol (200 mg), potassium carbonate (339 mg), 4-chlorobutanol (0.16 ml) and dimethylformamide (20 ml), to give 100 mg (43%) of the title compound as white solid.

¹H-NMR (400 MHz, CDCl₃) δ: 7.63 (d, 1H, J=16 Hz), 7.23 (m, 2H), 6.38 (d, 1H, J=16 Hz), 6.26 (s, 2H), 5.71 (m, 1H), 5.19 (m, 1H), 3.92 (m, 2H), 3.69 (dd, 1H, J=11, 2.7 Hz), 3.55 (m, 2H), 3.45 (s, 3H), 2.99 (d, 1H, J=4.4 Hz), 2.60 (t, 1H, J=6.4 Hz), 2.56 (d, 1H, J=4.4 Hz), 2.35 (m, 4H), 2.19-2.00 (m, 4H), 1.90 (m, 1H), 1.74 (s, 3H), 1.70 (m, 2H), 1.65 (s, 3H), 1.48 (m, 2H), 1.22 (s, 3H), 1.08 (m, 1H).

### Example 23

### A preparation of O-(4-(5-hydroxypentoxy)cinnamoyl)fumagillol

The same procedure as described in the step 3 of Example 1 was repeated but using O-(4-hydroxycinnamoyl)fumagillol (300 mg), 5-chloropentanol (0.32 ml), potassium carbonate (580 mg) and dimethylformamide (20 ml), to give 200 mg (56%) of the title compound as colorless syrup.

¹H-NMR (400 MHz, CDCl₃) δ: 7.61 (d, 1H, J=16 Hz), 7.20 (m, 2H), 6.71 (m, 2H), 6.35 (d, 1H, J=16 Hz), 5.72 (m, 1H), 5.21 (m, 1H), 3.92 (m, 2H), 3.69 (dd, 1H, J=11, 2.7 Hz), 3.53 (m, 2H), 3.44 (s, 3H), 2.98 (d, 1H, J=4.4 Hz), 2.61 (t, 1H, J=6.4 Hz), 2.56 (d, 1H, J=4.4 Hz), 2.35 (m, 1H), 2.18-2.01 (m, 4H), 1.91 (m, 1H), 1.75 (s, 3H), 1.71 (m, 2H), 1.64 (s, 3H), 1.48 (m, 2H), 1.29 (m, 2H), 1.22 (s, 3H), 1.07 (m, 1H).

### Example 24

### A preparation of O-(3-nitro-4-(5-hydroxypentoxy)cinnamoyl)fumagillol

The same procedure as described in the step 3 of Example 1 was repeated but using O-(3-nitro-4-hydroxycinnamoyl)fumagillol (500 mg), potassium carbonate (849 mg), 5-chloropentanol (0.47 ml) and dimethylformamide (20 ml), to give 300 mg (51%) of the title compound as colorless syrup.

¹H-NMR (400 MHz, CDCl₃) δ: 7.64 (d, 1H, J=16 Hz), 7.23 (m, 2H), 6.35 (d, 1H, J=16 Hz), 6.26 (s, 1H), 5.73 (m, 1H), 5.21 (m, 1H), 3.95 (m, 2H), 3.68 (dd, 1H, J=11, 2.7 Hz), 3.52 (m, 2H), 3.44 (s, 3H), 2.99 (d, 1H, J=4.4 Hz), 2.60 (t, 1H, J=6.4 Hz), 2.55 (d, 1H, J=4.4 Hz), 2.34 (m, 1H), 2.18-2.02 (m, 4H), 1.91 (m, 1H), 1.76-1.70 (m, 5H), 1.65 (s, 3H), 1.65 (s, 3H), 1.50 (m, 2H), 1.30 (m, 2H), 1.22 (s, 3H), 1.08 (m, 1H).

### Example 25

### A preparation of O-(4-(6-hydroxyhexyloxy)cinnamoyl)fumagillol

The same procedure as described in the step 3 of Example 1 was repeated but using O-(4-hydroxycinnamoyl)fumagillol (100 mg), potassium carbonate (194 mg), 6-chlorohexanol (0.12 ml) and dimethylformamide (10 ml), to give 50 mg (41%) of the title compound as colorless syrup.

¹H-NMR (400 MHz, CDCl₃) δ: 7.64 (d, 1H, J=16 Hz), 7.20 (m, 2H), 6.72 (m, 2H), 6.35 (d, 1H, J=16 Hz), 5.72 (m, 1H), 5.21 (m, 1H), 3.92 (m, 2H), 3.69 (dd, 1H, J=11, 2.7 Hz), 3.53 (m, 2H), 3.45 (s, 3H), 2.99 (d, 1H, J=4.4 Hz), 2.61 (t, 1H, J=6.4 Hz), 2.55 (d, 1H, J=4.4 Hz), 2.35 (m, 1H), 2.19-2.00 (m, 4H), 1.90 (m, 1H), 1.74 (s, 3H), 1.71 (m, 2H), 1.65 (s, 3H), 1.48 (m, 2H), 1.29 (m, 4H), 1.22 (s, 3H), 1.08 (m, 1H).

### Example 26

### A preparation of O-(3-trifluoromethyl-4-(6-hydroxyhexyloxy)cinnamoyl)fumagillol

The same procedure as described in the step 3 of Example 1 was repeated but using O-(3,5-dimethoxy-4-hydroxycinnamoyl)fumagillol (200 mg), potassium carbonate (339 mg), 6-chlorohexanol (0.22 ml) and dimethylformamide (10 ml), to give 70 mg (29%) of the title compound as colorless syrup.

¹H-NMR (400 MHz, CDCl₃) δ: 7.62 (d, 1H, J=16 Hz), 7.23 (m, 2H), 6.36 (d, 1H, J=16 Hz), 6.26 (m, 1H), 5.72 (m, 1H), 5.21 (m, 1H), 3.95 (m, 2H), 3.68 (dd, 1H, J=11, 2.7 Hz), 3.54 (m, 2H), 3.45 (s, 3H), 2.98 (d, 1H, J=4.4 Hz), 2.59 (t, 1H, J=6.4 Hz), 2.55 (d, 1H, J=4.4 Hz), 2.36 (m, 1H), 2.17-2.00 (m, 4H), 1.94 (m, 1H), 1.78 (s, 3H), 1.73 (m, 2H), 1.64 (s, 3H), 1.48 (m, 2H), 1.29 (m, 4H), 1.22 (s, 3H), 1.09 (m, 1H).

### Example 27

### A preparation of O-(4-(2-hydroxyethoxyethoxyethoxy)cinnamoyl)fumagillol

The same procedure as described in the step 3 of Example 1 was repeated but using O-(4-hydroxycinnamoyl)fumagillol (400 mg), potassium carbonate (774 mg), 2-(2-(2-chloroethoxy)ethoxy)ethanol (0.54 ml) and dimethylformamide (20 ml), to give 400 mg (76%) of the title compound as colorless syrup.

¹H-NMR (400 MHz, CDCl₃) δ: 7.66 (d, 1H, J=16 Hz), 7.46 (m, 2H), 6.89 (m, 2H), 6.35 (d, 1H, J=16 Hz), 5.73 (m, 1H), 5.20 (m, 1H), 4.11 (m, 2H), 3.96 (m, 2H), 3.69 (m, 3H), 3.54 (m, 6H), 3.45 (s, 3H), 2.99 (d, 1H, J=4.4 Hz), 2.60 (t, 1H, J=6.4 Hz), 2.56 (d, 1H, J=4.4 Hz), 2.35 (m, 1H), 2.20-1.88 (m, 5H), 1.74 (s, 3H), 1.65 (s, 3H), 1.24 (s, 3H), 1.11 (m, 1H).

### Example 28

### A preparation of O-(4-(2-hydroxyethylamino)cinnamoyl)fumagillol maleate

O-(4-(2-hydroxyethylamino)cinnamoyl)fumagillol (200 mg, 0.424 mmol) was dissolved in methanol (10 ml), maleic acid (49 mg, 0.424 mmol) was added thereto, and the solution was stirred for 1 hour at ordinary temperature. Solvent was concentrated under reduced pressure and was dried under the vacuum to give 240 mg (96%) of the title compound as white solid.

¹H-NMR (400 MHz, CDCl₃) δ: 7.57 (d, 1H, J=16 Hz), 7.34 (m, 2H), 6.74 (m, 2H), 6.44 (d, 1H, J=16 Hz), 6.37 (m, 2H), 5.74 (m, 1H), 5.21 (m, 1H), 3.87 (m, 2H), 3.70 (m, 1H), 3.47 (s, 3H), 3.36 (m, 2H), 3.00 (d, 1H, J=4 Hz), 2.63 (m, 1H), 2.57 (d, 1H, J=4 Hz), 2.40 (m, 1H), 2.20-1.88 (m, 5H), 1.74 (s, 3H), 1.66 (s, 3H), 1.25 (s, 3H), 1.11 (m, 1H).

The structures of the compounds according to Examples 1˜27 were represented in Table 1.

### Pharmaceutical Preparation Example 1

### Preparation of Tablet

| | |
|---|---|
| O-(4-(2-hydroxyethoxy)cinnamoyl)fumagillol: | 5.0 mg |
| Lactose BP: | 150.0 mg |
| Starch BP: | 30.0 mg |
| Pregelatinized corn starch BP: | 15.0 mg |
| Magnesium stearate: | 1.0 mg |

O-(4-(2-hydroxyethoxy)cinnamoyl)fumagillol (a compound of the Example 1) was sieved, and mixed with lactose, starch and pregelatinized corn starch. Purified water was added thereto in appropriate amount and the mixture was granulated. The resultant granule was dried, mixed with magnesium stearate, and then compressed to obtain tablet.

### Pharmaceutical Preparation Example 2

### Preparation of Capsule

| | |
|---|---|
| O-(3,5-dimethoxy-4- | 5.0 mg |
| (2-hydroxyethoxy)cinnamoyl)fumagillol: | |
| Starch 1500: | 100.0 mg |
| Magnesium stearate BP: | 1.0 mg |

O-(3,5-dimethoxy-4-(2-hydroxyethoxy)cinnamoyl)fumagillol (a compound of the Example 2) was sieved, and mixed with excipients. This mixture was then filled into gelatin capsule to give the capsule.

### Pharmaceutical Preparation Example 3

### Preparation of Capsule

| | |
|---|---|
| O-(4-(2-hydroxyethoxy)cinnamoyl)fumagillol: | 5.0 mg |
| Hydroxypropyl-β-cyclodextrin: | 50.0 mg |
| Starch 1500: | 100.0 mg |
| Magnesium stearate BP: | 1.0 mg |

O-(4-(2-hydroxyethoxy)cinnamoyl)fumagillol (a compound of the Example 1) and hydroxypropyl-β-cyclodextrin were dissolved in water, dried and sieved, to give inclusion complex powder. After this inclusion complex was mixed with leftover excipients, it was filled into gelatin capsule to give the capsule.

### Pharmaceutical Preparation Example 4

### Preparation of Injection

| O-(4-(2-hydroxyethoxy)-3- | 100 | μg/ml |
|---|---|---|
| methoxycinnamoyl)fumagillol: | | |
| Diluted Hydrochloric acid BP: | to be pH 3.5 | |
| Sodium chloride BP for injection: | maximum 1 | ml |

O-(4-(2-hydroxyethoxy)-3-methoxycinnamoyl)fumagillol (a compound of the Example 3) was dissolved in appropriate volume of sodium chloride BP for injection. The pH of the resultant solution was regulated to be pH 3.5 with d-HCl BP, and then its volume was controlled with sodium chloride BP for Injection and the solution was mixed completely. The solution was then filled into 5-ml type 1 ample made of transparent glass. The air was sealed in upper lattice by melting the glass. The solution contained in ample was autoclaved at 120° C. for 15 min or more to be sterilized and thereby to obtain a preparation of injection.

### Pharmaceutical Preparation Example 5

### Preparation of Injection

| O-(4-(2-hydroxyethoxy)cinnamoyl)fumagillol: | 100 | μg/ml |
|---|---|---|
| Sulfobutylether-7-β-cyclodextrin: | 500 | μg/ml |
| Diluted Hydrochloric acid BP: | to be pH 3.5 | |
| Sodium chloride BP for injection: | maximum 1 | ml |

O-(4-(2-hydroxyethoxy)cinnamoyl)fumagillol (a compound of the Example 1) and sulfobutylether-7-β-cyclodextrin was dissolved in appropriate volume of sodium chloride BP for injection. The pH of the resultant solution was regulated to be pH 3.5 with d-HCl BP, and then its volume was regulated with sodium chloride BP for Injection and the solution was mixed completely. The solution was then filled in 5-ml type 1 ample that is made of transparent glass. The air was sealed in upper lattice by melting the glass. The solution contained in ample was autoclaved at 120° C. for 15 min or more to be sterilized and thereby to obtain an injection.

### Experimental Example 1

### Examination of the Inhibiting Activity on Cell Proliferation (In Vitro)

### (1) Cell Line Culture

In order to compare the safety index (SI) of CPAE (calf pulmonary artery endothelial cell) with that of HUVEC (human umbilical vein endothelial cell), L5178Y lymphoma that was isolated from murine thymus was used. The safety index was defined as IC₅₀ ratio of L5178Y against CPAE (IC_{50L5178Y}/IC_{50CPAE}). All cell lines, which had been kept in liquid nitrogen tank, were used after being thawed and subcultured 2˜3 times in T-flask. CPAE, HUVEC and L5178Y were cultured in MEM culture medium (20% FBS, 50˜100 μg/ml ECGS, 0.15% baking soda, 0.05 mg/ml gentamicin), M199 culture medium (20% FBS, 0.22% baking soda, 100 μg/ml heparin, 3 ng/ml bFGF, 0.05 mg/ml gentamicin) and RPMI 1640 culture medium (10% FBS, 0.2% baking soda, 0.05 mg/ml gentamicin), respectively, under the condition of 37° C. and 5% CO₂.

### (2) Cell Inoculation and Drug Treatment

The drug was prepared by being gradationally diluted to two or ten times by using PBS, 20 μl of the solution was added to each well of 96 well plate in triplicate. The cell, which is being incubated, was treated with trypsin to give cell suspension. The number of cell thereafter was counted. And 180 μl of the solution was inoculated to each well and cultured.

### (3) SRB Analysis (CPAE, HUVEC)

The cell was cultured with drugs for 3 days and then 50 μl of 50% TCA was added thereto (final concentration 10%). Subsequently the cell was fixed by being left alone at 4° C. for 1 hour. The well was washed 4 times by distilled water and then dried. After that 100 μl of SRB (Sulforhodamine B, Sigma Chemical Co.) solution (0.4% w/v in 1% acetic acid) was added thereto and then it was left alone at ordinary temperature for 30 minutes. Thereafter the well was washed 4 times by 1% acetic acid and dried. And then after adding 200 μl of 10 mM tris buffer, the absorbance at 570 nm was measured by automatic microplate reader (Model: Elx 808, Bio-Tek Instrument, INC). The viability was calculated from the ratio of the absorbance of the control to which drugs were not added and the well to which drugs were added. And the drug concentration that shows 50% of viability was provided in Table 2 as IC₅₀.

### (4) MTT Analysis (L5178Y)

The tumor cell was cultured with drugs for 3 days and then 50 μl of MTT (3-(4,5-dimethylthiazole-2-yl)-2,5-diphenyl tetrazolium bromide, Sigma Chemical Co.) solution (2.5 mg/ml in PBS) was added thereto. And it was further cultured for 4 hours at 37° C. Culture medium was removed carefully. And after dissolving formazan crystal by adding 150 μl of DMSO, the absorbance at 570 nm was determined. And IC₅₀ value was calculated by the same method as described in SRB analysis and was provided in Table 2.

TABLE 2

| | IC₅₀ (nM) | | | Selectivity Index |
|---|---|---|---|---|
| Compound | CAPE | HUVEC | L5178Y | (L5178Y/CAPE) |
| TNP-470¹⁾ | 4.49 × 10⁻¹ | 8.96 × 10⁻³ | 4304 | 3.85 × 10³ |
| CKD-732²⁾ | 6.48 × 10⁻¹ | 6.60 × 10⁻³ | 8721 | 1.35 × 10⁴ |
| CKD-731³⁾ | 4.4 × 10⁻³ | 5.60 × 10⁻⁴ | 58157 | 1.30 × 10⁷ |
| Example 1 | 2.5 × 10⁻³ | 9.10 × 10⁻⁶ | 15532 | 6.22 × 10⁶ |
| Example 2 | 2.5 × 10⁻³ | 1.36 × 10⁻⁵ | 25027 | 1.00 × 10⁷ |
| Example 3 | 4.5 × 10⁻³ | 1.74 × 10⁻⁵ | 56386 | 1.26 × 10⁷ |
| Example 4 | 6.0 × 10⁻³ | 3.76 × 10⁻³ | 27298 | 4.53 × 10⁶ |
| Example 5 | 6.1 × 10⁻³ | 1.04 × 10⁻³ | 7110 | 1.16 × 10⁶ |
| Example 6 | 7.1 × 10⁻³ | 2.22 × 10⁻³ | 27670 | 3.88 × 10⁶ |
| Example 7 | 6.5 × 10⁻³ | 1.70 × 10⁻³ | 31010 | 4.77 × 10⁶ |
| Example 8 | 8.57 × 10⁻¹ | 4.56 × 10⁻³ | 60190 | 7.03 × 10⁴ |
| Example 9 | 8.11 × 10⁻¹ | 3.61 × 10⁻³ | 61200 | 7.54 × 10⁴ |
| Example 10 | 7.11 × 10⁻¹ | 3.10 × 10⁻⁴ | 42530 | 6.00 × 10⁴ |
| Example 11 | 6.55 × 10⁻¹ | 3.60 × 10⁻³ | 75010 | 1.15 × 10⁵ |
| Example 12 | 1.9 × 10⁻² | 6.50 × 10⁻⁵ | 20516 | 1.08 × 10⁶ |
| Example 13 | 1.7 × 10⁻¹ | 7.6 × 10⁻⁵ | 34570 | 2.03 × 10⁵ |
| Example 14 | 2.6 × 10⁻¹ | 3.5 × 10⁻⁴ | 50140 | 1.93 × 10⁵ |
| Example 15 | 4.0 × 10⁻¹ | 3.8 × 10⁻⁴ | 41870 | 1.05 × 10⁵ |
| Example 16 | 4.1 × 10⁻² | 2.2 × 10⁻³ | 9486 | 2.31 × 10⁵ |
| Example 17 | 4.9 × 10⁻² | 3.02 × 10⁻³ | 30670 | 6.26 × 10⁵ |
| Example 18 | 4.5 × 10⁻² | 1.9 × 10⁻³ | 41877 | 9.31 × 10⁵ |
| Example 19 | 1.7 × 10⁻² | 8.6 × 10⁻⁵ | 24876 | 1.46 × 10⁶ |
| Example 20 | 4.6 × 10⁻² | 3.5 × 10⁻⁴ | 20457 | 4.45 × 10⁵ |
| Example 21 | 2.9 × 10⁻² | 3.7 × 10⁻⁵ | 25800 | 8.90 × 10⁵ |
| Example 22 | 4.8 × 10⁻² | 8.9 × 10⁻⁴ | 32470 | 6.76 × 10⁵ |
| Example 23 | 4.67 × 10⁻² | 5.7 × 10⁻³ | 9250 | 1.98 × 10⁵ |
| Example 24 | 9.46 × 10⁻² | 4.2 × 10⁻³ | 10427 | 1.10 × 10⁵ |
| Example 25 | 1.24 × 10⁻¹ | 1.5 × 10⁻³ | 21057 | 1.70 × 10⁵ |
| Example 26 | 6.77 × 10⁻¹ | 1.9 × 10⁻³ | 9987 | 1.48 × 10⁴ |
| Example 27 | 8.9 × 10⁻² | 3.86 × 10⁻⁴ | 10524 | 1.18 × 10⁵ |

¹⁾TNP-470: O-chloroacetylcarbamoylfumagillol (refer to EP B1-357061)
²⁾CKD-732: O-(4-dimethylaminoethoxycinnamoyl)fumagillol (refer to US 6063812A)
³⁾CKD-731: O-(3,4,5-trimethoxycinnamoyl)fumagillol (refer to US 6063812A)

As can be seen from the result of the Table 2, the compounds of the present invention have more excellent inhibiting activity on cell proliferation than CKD-731 that is known as the compound having the most excellent inhibiting activity on cell proliferation out of all publicly known compounds. Particularly, a compound of the Examples 1, 2 and 3 show a equivalent or not smaller than twice effect of CKD-731 against CPAE, a compound of the Examples 1, 2, 3, 12 and 21 shows not smaller than 10˜100 times effect of CKD-731 against HUVEC, and a compound of the Examples 2 and 3 shows a equivalent or more effect of CKD-731 against SI. From these results, it was confirmed that the compounds of the present invention strongly inhibit the proliferation of hemangioendothelioma, and that the compounds of the present invention can be used as angiogenesis inhibitor.

### Experimental Example 2

### The Acute Oral Toxicity Test on Rats (In Vivo)

In order to test the acute toxicity of the compounds of the present invention, the experiment as set forth below was carried out.

The acute toxicity test was carried out using 6-weeks-old SD rats. Each Example of O-(4-(2-hydroxyethoxy)cinnamoyl)fumagillol (a compound of the Example 1), O-(3,5-dimethoxy-4-(2-hydroxyethoxy)cinnamoyl)fumagillol (a compound of the Example 2) and O-(4-(2-hydroxyethoxy)-3-methoxycinnamoyl)fumagillol (a compound of the Example 3) was suspended in 0.5% methylcellulose, and then orally administrated once to 5 of each male and each female rats per group by dosage of 1 g/kg/15 ml. After administrating the test substances, the death of the rats, clinic symptoms and the change of weight were observed. And also hematologic and blood biochemical tests were executed, the abnormality of abdominal and thoracic organs was observed by autopsy with the naked eye. As a result, there were no notable clinic symptoms, death and change by toxicity, when observed by autopsy, in all the rats to which the test substances were administrated.

As a result of the above test, the compounds of the present invention did not show any change of toxicity in all rats until its dosage reached 2 g/kg, and it was estimated that the compounds of the present invention is the safe compounds, oral administration Lethal Dose 50% (LD₅₀) of which is not less than 2 g/kg.

### Solubility Test

To a constant amount (400 mg) of each compounds of present invention, a constant amount of demineralized water, methanol and ethanol were added separately, and the solution was stirred at ordinary temperature. The solubility of each compound is provided in Table 3.

TABLE 3

| | The amount of solvent to dissolve | | |
|---|---|---|---|
| | 1 g of the compounds (ml) | | |
| Compound | demineralized water | methanol | ethanol |
| TNP-470 | 876 | 208 | 250 |
| CKD-732 | 1100 | 197 | 219 |
| CKD-731 | 1567 | 350 | 372 |
| Example 1 | 135 | 34 | 55 |
| Example 2 | 146 | 37 | 59 |
| Example 3 | 142 | 45 | 61 |
| Example 4 | 145 | 49 | 65 |
| Example 5 | 132 | 39 | 60 |
| Example 6 | 130 | 31 | 58 |
| Example 7 | 137 | 42 | 67 |
| Example 12 | 126 | 35 | 51 |
| Example 21 | 150 | 70 | 97 |
| Example 27 | 120 | 30 | 50 |

As can be seen from the result of the Table 3, the compounds of the present invention shows not less than 5˜13 times solubility in demineralized water, methanol, and ethanol, as compared to TNP-470, CKD-732 and CKD-731, which are hitherto known compounds. According to this result, it is considered that the compound of the present invention is excellently absorbed into body and accordingly the effective dose of drugs may be reduced.

### Chemical Stability Test

Each compound of the present invention was kept in an air-tight vessel for 1 month at 40±2° C. and 75±5% of relative humidity. Thereafter the HPLC purity test was executed.

(1) The Preparation of Test Liquid:

The publicly known compounds and the compounds of examples 1, 2, 3, 4, 5, 6, 7, 12, 21 and 27 were precisely weighed by 30 mg, poured into 100 mL volumetric flask, dissolved by adding acetonitrile/20 mM ammonium acetate aqueous solution (50:50), to be the total volume of 100 ml. 25 mL of the solution was precisely taken, and poured into 100 mL volumetric flask. Acetonitrile/20 mM ammonium acetate aqueous solution (50:50) was added thereto to be the total volume of 100 ml. The solution was filtrated, and the filtrate was used as a test liquid.

(2) Operational Condition of Instrument.
Column: Kromasil®C₁₈ (UG 100 Å, 5 μm, 4.6 mm Φ×250 mm)
Column temperature: 30° C.
Mobil phase: 20 mM ammonium acetate (pH 4.2) buffer:acetonitrile (55:45)
Injection volume: 20 μL
Flow rate: 1.2 mL/min
Detector: UV light-absorption Spectrophotometer (detection wavelength: 306 nm)

TABLE 4

| | Initial | | 1 month | | |
|---|---|---|---|---|---|
| | Parent | | Parent | | change of |
| | peak | Other peaks | peak | Other peaks | purity |
| Compounds | (%) | (%) | (%) | (%) | (%) |
| TNP-470 | 98.7 | 1.3 | 96.5 | 3.5 | 2.2 |
| CKD-732 | 99.2 | 0.8 | 95.4 | 4.6 | 3.8 |
| CKD-731 | 98.9 | 1.1 | 97.1 | 2.9 | 1.8 |
| Example 1 | 99.4 | 0.6 | 99.4 | 0.6 | 0 |
| Example 2 | 99.1 | 0.9 | 99.1 | 0.9 | 0 |
| Example 3 | 98.5 | 1.5 | 98.4 | 1.6 | 0.1 |
| Example 4 | 98.7 | 1.3 | 98.5 | 1.5 | 0.2 |
| Example 5 | 99.2 | 0.8 | 98.5 | 1.5 | 0.7 |
| Example 6 | 98.6 | 1.4 | 98.1 | 1.9 | 0.5 |
| Example 7 | 99.4 | 0.6 | 98.9 | 1.1 | 0.5 |
| Example 12 | 99.5 | 0.5 | 99.4 | 0.6 | 0.1 |
| Example 21 | 99.0 | 1.0 | 98.9 | 1.1 | 0.1 |
| Example 27 | 98.4 | 1.6 | 98.4 | 1.6 | 0 |

As can be seen from the result of the Table 4, compounds of the Example 1, 2, 3, 4, 5, 6, 7, 12, 21 and 27 showed more excellent chemical stability than TNP-470, CKD-732 and CKD-731. And particularly a compound of the example 1, 2, 3, 4, 12, 21 and 27 hardly showed a change of purity. From this result, the compounds of the present invention were estimated to be very stable compounds under above condition.

### INDUSTRIAL APPLICABILITY

The compounds of the present invention have 3 of strong points, as compared to the above-mentioned compounds that are publicly known.

First, the compounds of the present invention have a broad therapeutic range, low toxicity and excellent stability, as well as it can inhibit and reduce the growth and metastasis of cancer superiorly by inhibiting the growth of blood vessel endothelial cells.

Second, the compounds of the present invention are easily absorbed into body, since the solubility in demineralized water, methanol and ethanol is high. And thereby the effective dose of drugs may be reduced.

Third, the compounds of the present invention are estimated to be very stable compounds with respect to chemical stability.

Accordingly, the compounds of the Chemical Formula 1 can be used as angiogenesis inhibitors.

TABLE 1

| | The structure of compound |
|---|---|
| Example 1 | 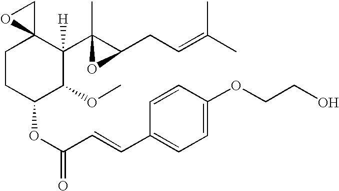 |
| Example 2 | 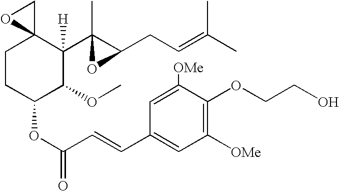 |
| Example 3 | 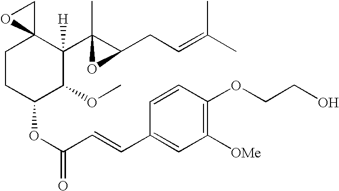 |
| Example 4 | 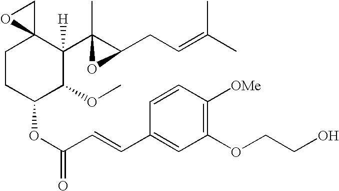 |
| Example 5 | 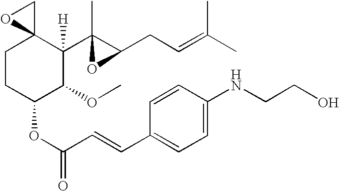 |
| Example 6 | 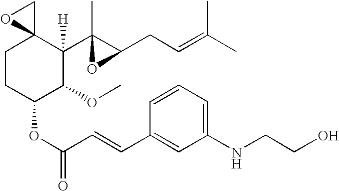 |
| Example 7 | 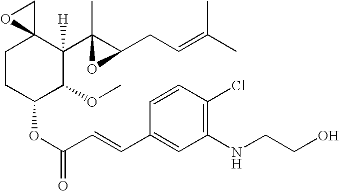 |
| Example 8 | 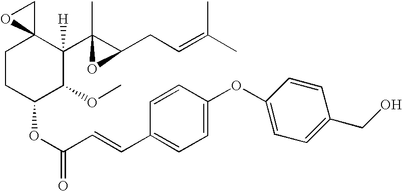 |
| Example 9 | 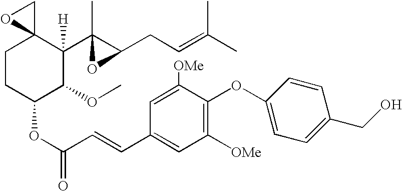 |
| Example 10 | 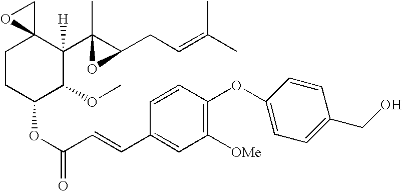 |
| Example 11 | 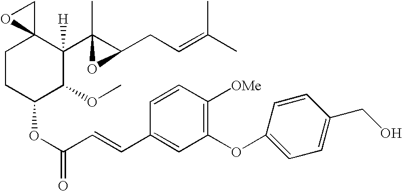 |
| Example 12 | 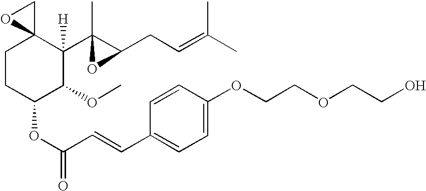 |
| Example 13 | 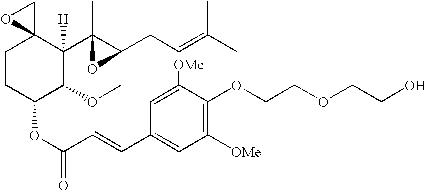 |
| Example 14 | 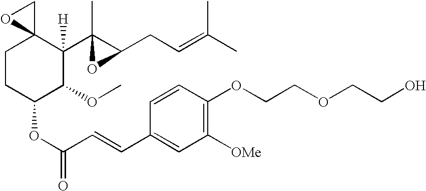 |
| Example 15 | 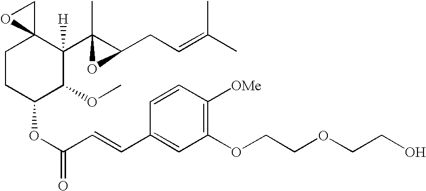 |
| Example 16 | 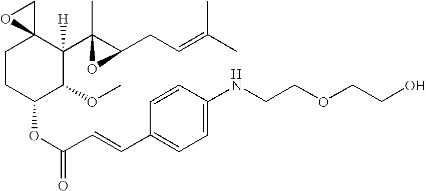 |
| Example 17 | 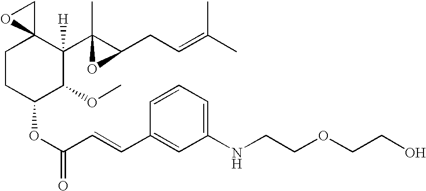 |
| Example 18 | 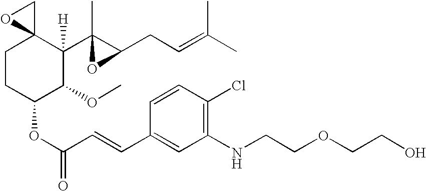 |
| Example 19 | 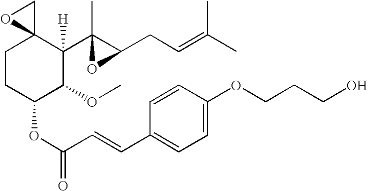 |
| Example 20 | 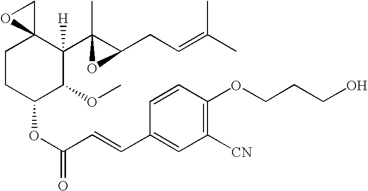 |
| Example 21 | 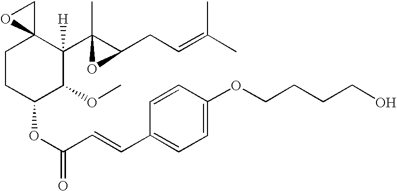 |
| Example 22 | 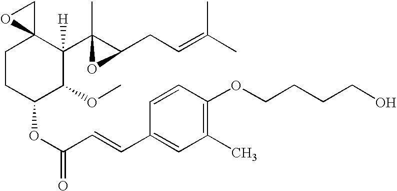 |
| Example 23 | 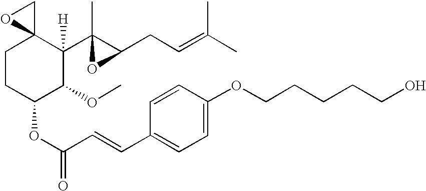 |
| Example 24 | 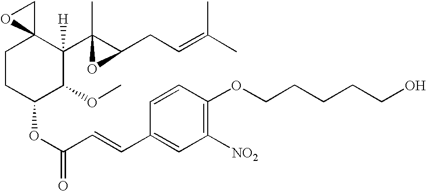 |
| Example 25 | 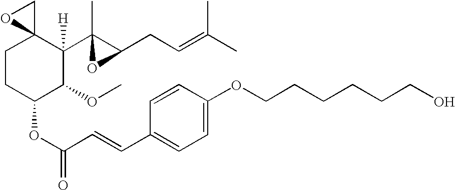 |
| Example 26 | 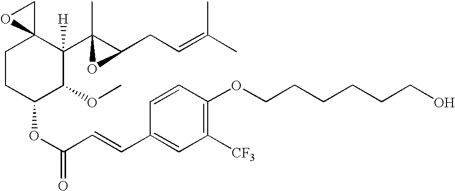 |
| Example 27 | 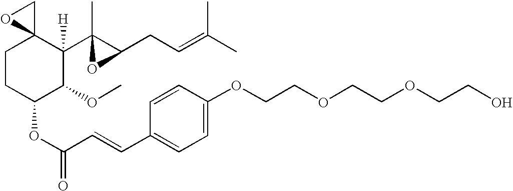 |

1. A fumagillol derivative of Formula 1 or a pharmaceutically acceptable salt thereof: 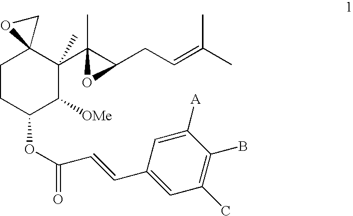
wherein, A, B and C represent independently or simultaneously hydrogen, C₁-C₆ alkoxy, halogen, C₁-C₆ alkyl, trifluoromethyl, cyano, nitro, 4-hydroxymethylphenoxy, —XCH₂ₙOH or —XCH₂CH₂OₘCH₂CH₂O, wherein X represents nitrogen or oxygen; n is 3, 4, 5 or 6; and m is 0, 1 or 2,
with proviso that at least one of above A, B, C is one substituent selected from 4-hydroxymethylphenoxy, —XCH₂ₙOH and —XCH₂CH₂OₘCH₂CH₂OH.)

2. The fumagillol derivative according to claim 1, which is selected from the group consisting of:
O-(4-(2-hydroxyethoxy)cinnamoyl)fumagillol,
O-(3,5-dimethoxy-4-(2-hydroxyethoxy)cinnamoyl)fumagillol,
O-(4-(2-hydroxyethoxy)-3-methoxycinnamoyl)fumagillol,
O-(3-(2-hydroxyethoxy)-4-methoxycinnamoyl)fumagillol,
O-(4-(2-hydroxyethylamino)cinnamoyl)fumagillol,
O-(3-(2-hydroxyethylamino)cinnamoyl)fumagillol,
O-(4-chloro-3-(2-hydroxyethylamino)cinnamoyl)fumagillol,
O-(4-(4-hydroxymethylphenoxy)cinnamoyl)fumagillol,
O-(3,5-dimethoxy-4-(4-hydroxymethylphenoxy)cinnamoyl) fumagillol,
O-(4-(4-hydroxymethylphenoxy)-3-methoxycinnamoyl)fumagillol,
O-(3-(4-hydroxymethylphenoxy)-4-methoxycinnamoyl)fumagillol,
O-(4-(2-hydroxyethoxyethoxy)cinnamoyl)fumagillol,
O-(3,5-dimethoxy-4-(2-hydroxyethoxyethoxy)cinnamoyl)fumagillol,
O-(4-(2-hydroxyethoxyethoxy)-3-methoxycinnamoyl)fumagillol,
O-(3-(2-hydroxyethoxyethoxy)-4-methoxycinnamoyl)fumagillol,
O-(4-(2-hydroxyethoxyethylamino)cinnamoyl)fumagillol,
O-(3-(2-hydroxyethoxyethylamino)cinnamoyl)fumagillol,
O-(4-chloro-3-(2-hydroxyethoxyethylamino)cinnamoyl)fumagillol,
O-(4-(3-hydroxypropoxy)cinnamoyl)fumagillol,
O-(3-cyano-4-(3-hydroxypropoxy)cinnamoyl)fumagillol,
O-(4-(4-hydroxybutoxy)cinnamoyl)fumagillol,
O-(3-methyl-4-(4-hydroxybutoxy)cinnamoyl)fumagillol,
O-(4-(5-hydroxypentoxy)cinnamoyl)fumagillol,
O-(3-nitro-4-(5-hydroxypentoxy)cinnamoyl)fumagillol,
O-(4-(6-hydroxyhexyloxy)cinnamoyl)fumagillol,
O-(3-trifluoromethyl-4-(6-hydroxyhexyloxy)cinnamoyl)fumagillol, and
O-(4-(2-hydroxyethoxyethoxyethoxy)cinnamoyl)fumagillol.

3. The fumagillol derivative according to claim 2, which is selected from the group consisting of:
O-(4-(2-hydroxyethoxy)cinnamoyl)fumagillol,
O-(3,5-dimethoxy-4-(2-hydroxyethoxy)cinnamoyl)fumagillol,
O-(4-(2-hydroxyethoxy)-3-methoxycinnamoyl)fumagillol,
O-(3-(2-hydroxyethoxy)-4-methoxycinnamoyl)fumagillol,
O-(4-(2-hydroxyethylamino)cinnamoyl)fumagillol,
O-(3-(2-hydroxyethylamino)cinnamoyl)fumagillol,
O-(4-chloro-3-(2-hydroxyethylamino)cinnamoyl)fumagillol, and
O-(4-(3-hydroxypropoxy)cinnamoyl)fumagillol.

4. A salt of the fumagillol derivative according to claim 1, wherein the pharmaceutically acceptable salt is hydrochloride, bromate, sulfate, phosphate, nitrate, citrate, acetate, lactate, tartarate, maleate, gluconate, succinate, formate, trifluoroacetate, oxalate, fumarate, methanesulfonate, benzenesulfonate, p-toluenesulfonate or campursulfonate salt.

5. A method for preparing a fumagillol derivative of the Chemical Formula 1, comprising alkylating a compound of the Chemical Formula 5 with a compound of the Chemical Formula 6 or a compound of the Chemical Formula 7 in the presence of 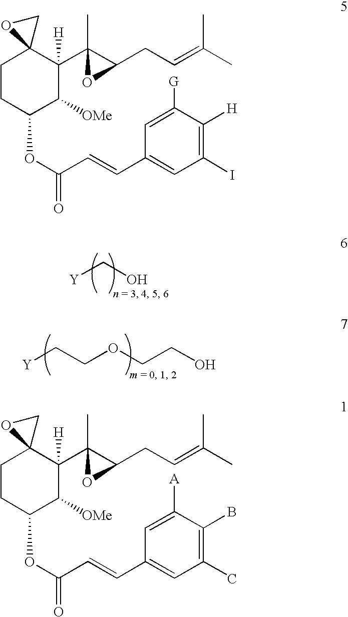
base.
wherein, A, B and C are the same as defined in claim 1; Y represents halogen; n is 3, 4, 5 or 6; m is 0, 1 or 2; and G, H and I represent independently or simultaneously hydrogen, C₁˜C₆ alkoxy, halogen, C₁˜C₆ alkyl, trifluoromethyl, cyano, nitro, 4-hydroxymethylphenoxy, hydroxy or amine, with proviso that at least one of above G, H, I is one substituent selected from 4-hydroxymethylphenoxy, hydroxy and amine.

6. The method according to claim 5, wherein the fumagillol derivative is selected from a group consisting of:
O-(4-(2-hydroxyethoxy)cinnamoyl)fumagillol,
O-(3,5-dimethoxy-4-(2-hydroxyethoxy)cinnamoyl)fumagillol,
O-(4-(2-hydroxyethoxy)-3-methoxycinnamoyl)fumagillol,
O-(3-(2-hydroxyethoxy)-4-methoxycinnamoyl)fumagillol,
O-(4-(2-hydroxyethylamino)cinnamoyl)fumagillol,
O-(3-(2-hydroxyethylamino)cinnamoyl)fumagillol,
O-(4-chloro-3-(2-hydroxyethylamino)cinnamoyl)fumagillol,
O-(4-(2-hydroxyethoxyethoxy)cinnamoyl)fumagillol,
O-(3,5-dimethoxy-4-(2-hydroxyethoxyethoxy)cinnamoyl)fumagillol,
O-(4-(2-hydroxyethoxyethoxy)-3-methoxycinnamoyl)fumagillol,
O-(3-2-hydroxyethoxyethoxy)-4-methoxycinnamoyl)fumagillol,
O-(4-(2-hydroxyethoxyethylamino)cinnamoyl)fumagillol,
O-(3-(2-hydroxyethoxyethylamino)cinnamoyl)fumagillol,
O-(4-chloro-3-(2-hydroxyethoxyethylamino)cinnamoyl)fumagillol,
O-(4-(3-hydroxypropoxy)cinnamoyl)fumagillol,
O-(3-cyano-4-(3-hydroxypropoxy)cinnamoyl)fumagillol,
O-(4-(4-hydroxybutoxy)cinnamoyl)fumagillol,
O-(3-methyl-4-(4-hydroxybutoxy)cinnamoyl)fumagillol,
O-(4-(5-hydroxypentoxy)cinnamoyl)fumagillol
O-(3-nitro-4-(5-hydroxypentoxy)cinnamoyl)fumagillol,
O-(4-(6-hydroxyhexyloxy)cinnamoyl)fumagillol,
O-(3-trifluoromethyl-4-(6-hydroxyhexyloxy)cinnamoyl)fumagillol, and
O-(4-(2-hydroxyethoxyethoxyethoxy)cinnamoyl)fumagillol.

7. The method according to claim 5, wherein a compound of Formula 5 is obtained by hydrolyzing a compound of Formula 4 in the presence of base. 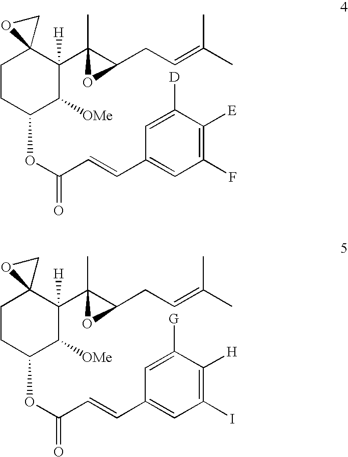
wherein, G, H and I are the same as defined in claim 5; and D, E and F represent independently or simultaneously hydrogen, C₁-C₆ alkoxy, halogen, C₁-C₆ alkyl, trifluoromethyl, cyano, nitro, acetoxy, acetamino or 4-acetoxymethylphenoxy, with proviso that at least one of above D, E, F is one substituent selected from acetoxy, acetamino and 4-acetoxymethylphenoxy)

8. An anti-tumor composition, which comprises a compound of Formula 1 of claim 1 or a pharmaceutically acceptable salt thereof as active ingredient, and a pharmaceutically acceptable carrier.

9. A pharmaceutical compositions comprising an inclusion compound, wherein the inclusion compound comprises a compound of Formula 1 of claim 1 or a pharmaceutically acceptable salt thereof, and hydroxypropyl-β-cyclodextrin or sulfobutylether-7-β-cyclodextrin.
